SEQUENCE LISTING (51 sequences)

SEQ ID NO: 1 - DNA, 1973 bp, Mus musculus
tgagcagcggggaggaggaagaggagactgctactgaaggcgacactgccaggggctctgggcatgtgggtccggcaggtaccctggtcattcacttgggctgtgctgcagttgagctggcaatcagggtggcttctagaggtccccaatgggccctggaggtccctcaccttctacccagcctggctcacagtgtcagagggagcaaatgccaccttcacctgcagcttgtccaactggtcggaggatcttatgctgaactggaaccgcctgagtcccagcaaccagactgaaaaacaggccgccttctgtaatggtttgagccaacccgtccaggatgcccgcttccagatcatacagctgcccaacaggcatgacttccacatgaacatccttgacacacggcgcaatgacagtggcatctacctctgtggggccatctccctgcaccccaaggcaaaaatcgaggagagccctggagcagagctcgtggtaacagagagaatcctggagacctcaacaagatatcccagcccctcgcccaaaccagaaggccggtttcaaggcatggtcattggtatcatgagtgccctagtgggtatccctgtattgctgctgctggcctgggccctagctgtcttctgctcaacaagtatgtcagaggccagaggagctggaagcaaggacgacactctgaaggaggagccttcagcagcacctgtccctagtgtggcctatgaggagctggacttccagggacgagagaagacaccagagctccctaccgcctgtgtgcacacagaatatgccaccattgtcttcactgaagggctgggtgcctcggccatgggacgtaggggctcagctgatggcctgcagggtcctcggcctccaagacatgaggatggacattgttcttggcctctttgaccagattcttcagccattagcatgctgcagaccctccacagagagcaccggtccgtccctcagtcaagaggagcatgcaggctacagttcagccaaggctcccagggtctgagctagctggagtgacagcccagcgcctgcaccaattccagcacatgcactgttgagtgagagctcacttcaggtttaccacaagctgggagcagcaggcttcccggtttcctattgtcacaaggtgcagagctggggcctaagcctatgtctcctgaatcctactgttgggcacttctagggacttgagacactatagccaatggcctctgtgggttctgtgcctggaaatggagagatctgagtacagcctgctttgaatggccctgtgaggcaaccccaaagcaagggggtccaggtatactatgggcccagcacctaaagccacccttgggagatgatactcaggtgggaaattcgtagactgggggactgaaccaatcccaagatctggaaaagttttgatgaagacttgaaaagctcctagcttcgggggtctgggaagcatgagcacttaccaggcaaaagctccgtgagcgtatctgctgtccttctgcatgcccaggtacctcagtttttttcaacagcaaggaaactagggcaataaagggaaccagcagagctagagccacccacacatccaggggggcacttgactctccctactcctcctaggaaccaaaaggacaaagtccatgttgacagcagggaaggaaagggggatataaccttgacgcaaaccaacactggggtgttagaatctcctcattcactctgtcctggagttgggttctggctctccttcacacctaggactctgaaatgagcaagcacttcagacagtcagggtagcaagagtctagctgtctggtgggcacccaaaatgaccagggcttaagtccctttcctttggtttaagcccgttataattaaatggtaccaaaagctttaaaaaaaaaaaaaaaaaaaaaaaaaaaaaaaaaaaaaaaaaa

SEQ ID NO: 2 - protein, 288 aa, Mus musculus
MWVRQVPWSFTWAVLQLSWQSGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMVIGIMSALVGIPVLLLLAWALAVFCSTSMSEARGAGSKDDTLKEEPSAAPVPSVAYEELDFQGREKTPELPTACVHTEYATIVFTEGLGASAMGRRGSADGLQGPRPPRHEDGHCSWPL

SEQ ID NO: 3 - DNA, 2115 bp, Homo sapiens
agtttcccttccgctcacctccgcctgagcagtggagaaggcggcactctggtggggctgctccaggcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaaggaaaa

SEQ ID NO: 4 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 5 - DNA, 408 bp, Artificial Sequence
  Description of Artificial Sequence Synthetic polynucleotide
atggaatggccttgtatctttctcttcctcctgtcagtaactgaaggtgtccactcccaggttcagctgcagcagtctggacctgagctggtgaagcctggggcctcagtgaagatttcctgcaaggcttctggcttcgcattcagtggctcctggatgaactggatgaagcagaggcctggaaagggtcttgagtggattggacggatttatcctggagatggagatactaactacaatgggaagtccaagggcaaggccacacttactgcagacacatcctccagcacagcctacatgcaactcagcagcctgacatctgaggactctgcggtctacttctgtacaagatcaactacgatattagcagactactggggccaaggcaccactctcacagtctcctca

SEQ ID NO: 6 - protein, 136 aa, Artificial Sequence
  Description of Artificial Sequence Synthetic polypeptide
MEWPCIFLFLLSVTEGVHSQVQLQQSGPELVKPGASVKISCKASGFAFSGSWMNWMKQRPGKGLEWIGRIYPGDGDTNYNGKSKGKATLTADTSSSTAYMQLSSLTSEDSAVYFCTRSTTILADYWGQGTTLTVSS

SEQ ID NO: 7 - DNA, 381 bp, Artificial Sequence
  Description of Artificial Sequence Synthetic polynucleotide
atgatgtcctctgctcagttccttggtctcctgttgctctgtttgcaaggtaccagatgtgatatccagatgacacagactacatcctccctgtctgcctctctgggagacagagtcaccatcagttgcagggcaagtcaggacattgccaattatttaaactggtatcagcagaaaccagatggaactgttaaactcctgatctactacacatcaagaaaatattcaggagtcccatcaaggttcagtggcagtgggtctggaacagattattctctcaccattagcaacctggaccaagaagatattgccacttacttttgccaacagggtaaaacgcttccgtggacgttcggtggaggcaccaagctggaaatcaaa

SEQ ID NO: 8 - protein, 127 aa, Artificial Sequence
  Description of Artificial Sequence Synthetic polypeptide
MMSSAQFLGLLLLCLQGTRCDIQMTQTTSSLSASLGDRVTISCRASQDIANYLNWYQQKPDGTVKLLIYYTSRKYSGVPSRFSGSGSGTDYSLTISNLDQEDIATYFCQQGKTLPWTFGGGTKLEIK

SEQ ID NO: 9 - protein, 10 aa, Homo sapiens
TDKLAAFPED

SEQ ID NO: 10 - protein, 30 aa, Artificial Sequence
  Description of Artificial Sequence Synthetic polypeptide
QVQLQQSGPELVKPGASVKISCKASGFAFS

SEQ ID NO: 11 - protein, 5 aa, Artificial Sequence
  Description of Artificial Sequence Synthetic peptide
GSWMN

SEQ ID NO: 12 - protein, 14 aa, Artificial Sequence
  Description of Artificial Sequence Synthetic peptide
WMKQRPGKGLEWIG

SEQ ID NO: 13 - protein, 17 aa, Artificial Sequence
  Description of Artificial Sequence Synthetic peptide
RIYPGDGDTNYNGKSKG

SEQ ID NO: 14 - protein, 32 aa, Artificial Sequence
  Description of Artificial Sequence Synthetic polypeptide
KATLTADTSSSTAYMQLSSLTSEDSAVYFCTR

SEQ ID NO: 15 - protein, 8 aa, Artificial Sequence
  Description of Artificial Sequence Synthetic peptide
STTILADY

SEQ ID NO: 16 - protein, 11 aa, Artificial Sequence
  Description of Artificial Sequence Synthetic peptide
WGQGTTLTVSS

SEQ ID NO: 17 - protein, 23 aa, Artificial Sequence
  Description of Artificial Sequence Synthetic peptide
DIQMTQTTSSLSASLGDRVTISC

SEQ ID NO: 18 - protein, 11 aa, Artificial Sequence
  Description of Artificial Sequence Synthetic peptide
RASQDIANYLN

SEQ ID NO: 19 - protein, 15 aa, Artificial Sequence
  Description of Artificial Sequence Synthetic peptide
WYQQKPDGTVKLLIY

SEQ ID NO: 20 - protein, 7 aa, Artificial Sequence
  Description of Artificial Sequence Synthetic peptide
YTSRKYS

SEQ ID NO: 21 - protein, 32 aa, Artificial Sequence
  Description of Artificial Sequence Synthetic polypeptide
GVPSRFSGSGSGTDYSLTISNLDQEDIATYFC

SEQ ID NO: 22 - protein, 9 aa, Artificial Sequence
  Description of Artificial Sequence Synthetic peptide
QQGKTLPWT

SEQ ID NO: 23 - protein, 10 aa, Artificial Sequence
  Description of Artificial Sequence Synthetic peptide
FGGGTKLEIK

SEQ ID NO: 24 - protein, 10 aa, Artificial Sequence
  Description of Artificial Sequence Synthetic peptide
PGWFLDSPDR

SEQ ID NO: 25 - protein, 10 aa, Artificial Sequence
  Description of Artificial Sequence Synthetic peptide
DSPDRPWNPP

SEQ ID NO: 26 - protein, 10 aa, Artificial Sequence
  Description of Artificial Sequence Synthetic peptide
PWNPPTFSPA

SEQ ID NO: 27 - protein, 10 aa, Artificial Sequence
  Description of Artificial Sequence Synthetic peptide
TFSPALLVVT

SEQ ID NO: 28 - protein, 10 aa, Artificial Sequence
  Description of Artificial Sequence Synthetic peptide
LLVVTEGDNA

SEQ ID NO: 29 - protein, 10 aa, Artificial Sequence
  Description of Artificial Sequence Synthetic peptide
EGDNATFTCS

SEQ ID NO: 30 - protein, 10 aa, Artificial Sequence
  Description of Artificial Sequence Synthetic peptide
TFTCSFSNTS

SEQ ID NO: 31 - protein, 10 aa, Artificial Sequence
  Description of Artificial Sequence Synthetic peptide
FSNTSESFVL

SEQ ID NO: 32 - protein, 10 aa, Artificial Sequence
  Description of Artificial Sequence Synthetic peptide
ESFVLNWYRM

SEQ ID NO: 33 - protein, 10 aa, Artificial Sequence
  Description of Artificial Sequence Synthetic peptide
NWYRMSPSNQ

SEQ ID NO: 34 - protein, 10 aa, Artificial Sequence
  Description of Artificial Sequence Synthetic peptide
SPSNQTDKLA

SEQ ID NO: 35 - protein, 10 aa, Artificial Sequence
  Description of Artificial Sequence Synthetic peptide
AFPEDRSQPG

SEQ ID NO: 36 - protein, 10 aa, Artificial Sequence
  Description of Artificial Sequence Synthetic peptide
RSQPGQDCRF

SEQ ID NO: 37 - protein, 10 aa, Artificial Sequence
  Description of Artificial Sequence Synthetic peptide
QDCRFRVTQL

SEQ ID NO: 38 - protein, 10 aa, Artificial Sequence
  Description of Artificial Sequence Synthetic peptide
RVTQLPNGRD

SEQ ID NO: 39 - protein, 10 aa, Artificial Sequence
  Description of Artificial Sequence Synthetic peptide
PNGRDFHMSV

SEQ ID NO: 40 - protein, 10 aa, Artificial Sequence
  Description of Artificial Sequence Synthetic peptide
FHMSVVRARR

SEQ ID NO: 41 - protein, 10 aa, Artificial Sequence
  Description of Artificial Sequence Synthetic peptide
VRARRNDSGT

SEQ ID NO: 42 - protein, 10 aa, Artificial Sequence
  Description of Artificial Sequence Synthetic peptide
NDSGTYLCGA

SEQ ID NO: 43 - protein, 10 aa, Artificial Sequence
  Description of Artificial Sequence Synthetic peptide
YLCGAISLAP

SEQ ID NO: 44 - protein, 10 aa, Artificial Sequence
  Description of Artificial Sequence Synthetic peptide
ISLAPKAQIK

SEQ ID NO: 45 - protein, 10 aa, Artificial Sequence
  Description of Artificial Sequence Synthetic peptide
KAQIKESLRA

SEQ ID NO: 46 - protein, 10 aa, Artificial Sequence
  Description of Artificial Sequence Synthetic peptide
ESLRAELRVT

SEQ ID NO: 47 - protein, 10 aa, Artificial Sequence
  Description of Artificial Sequence Synthetic peptide
ELRVTERRAE

SEQ ID NO: 48 - protein, 10 aa, Artificial Sequence
  Description of Artificial Sequence Synthetic peptide
ERRAEVPTAH

SEQ ID NO: 49 - protein, 10 aa, Artificial Sequence
  Description of Artificial Sequence Synthetic peptide
VPTAHPSPSP

SEQ ID NO: 50 - protein, 10 aa, Artificial Sequence
  Description of Artificial Sequence Synthetic peptide
PSPSPRPAGQ

SEQ ID NO: 51 - protein, 10 aa, Artificial Sequence
  Description of Artificial Sequence Synthetic peptide
RPAGQFQTLV

(19) United States
(12) Patent Application Publication
(10) Pub. No.: US 2021/0363255 A1
(43) Pub. Date: Nov. 25, 2021

(54) METHODS AND COMPOSITIONS FOR BLOCKING INTERACTION BETWEEN NON-GLYCOSYLATED PD-1 POLYPEPTIDES

(71) Applicant: TrueBinding, Inc., Foster City, CA (US)

(72) Inventors: Dongxu Sun, Los Altos, CA (US); Yan Wang, Concord, CA (US); Catherine A. Gordon, Fremont, CA (US); Samuel A.F. Williams, Burlingame, CA (US)

(21) Appl. No.: 17/041,280

(22) PCT Filed: Apr. 4, 2019
(86) PCT No.: PCT/US2019/025896

§ 371 Date: Sep. 24, 2020

Related U.S. Application Data
(60) Provisional application No. 62/652,857, filed on Apr. 4, 2018.

(51) Int. Cl. C07K 16/28; A61P 35/00; A61K 45/06

(52) U.S. Cl. CPC C07K 16/2818; A61K 2039/505; A61K 45/06; A61P 35/00

(57) ABSTRACT

Provided herein are methods and compositions for activating an immune response in a patient hosting a tumor. In some instances, a method described herein comprises administering to the patient a non-glycosylated PD-1 inhibitor and optionally a glycosylated PD-1 inhibitor, in which the non-glycosylated PD-1 inhibitor and optionally in combination with a glycosylated PD-1 inhibitor results in activation of the immune response.

### CROSS-REFERENCE

This application claims the benefit of U.S. Provisional Patent Application No. 62/652,857, filed on Apr. 4, 2018, which is incorporated herein by reference in its entirety.

### SUMMARY OF THE DISCLOSURE

Disclosed herein, in certain embodiments, are compositions and methods that impair or block the interaction between non-glycosylated PD-1 polypeptides that are located on the cell surface of different immune cells. In some instances, also disclosed herein are methods of suppressing T cell activation by impairing or blocking the interaction between two non-glycosylated PD-1 polypeptides.

In certain embodiments, disclosed herein is an antibody that impairs an interaction between two non-glycosylated PD-1 polypeptides, wherein the antibody comprises three variable heavy chain complementarity-determining regions (CDRs) and three variable light chain CDRs, and wherein the antibody specifically binds to a region of the non-glycosylated PD-1 polypeptide comprising SEQ ID NO: 9.

In certain embodiments, disclosed herein is a pharmaceutical combination comprising: a non-glycosylated PD-1 inhibitor that impairs an interaction between two non-glycosylated PD-1 polypeptides; a glycosylated PD-1 inhibitor that impairs an interaction between a glycosylated PD-1 polypeptide and a programmed cell death ligand; and a pharmaceutically acceptable vehicle or excipient.

In certain embodiments, disclosed herein is a method of disrupting an interaction between two non-glycosylated PD-1 polypeptides, comprising: contacting a non-glycosylated PD-1 inhibitor to a plurality of cells comprising two cells each expressing a non-glycosylated PD-1 polypeptide, wherein the non-glycosylated PD-1 inhibitor impairs the interaction between the two non-glycosylated PD-1 polypeptides.

In certain embodiments, disclosed herein is a method of activating an immune response in a subject in need thereof, comprising: administering to the subject a non-glycosylated PD-1 inhibitor and a glycosylated PD-1 inhibitor to activate the immune response, wherein the non-glycosylated PD-1 inhibitor impairs an interaction between two non-glycosylated PD-1 polypeptides, and wherein the glycosylated PD-1 inhibitor impairs an interaction between a glycosylated PD-1 polypeptide and a programmed cell death ligand.

In certain embodiments, disclosed herein is a method of reducing tumor cells within a tumor microenvironment (TME) in a subject, comprising: contacting a plurality of cells located within the TME with a non-glycosylated PD-1 inhibitor and a glycosylated PD-1 inhibitor.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-FIG. 1D show the results of co-immunoprecipitation assay by which it was shown that human PD-1 specifically pulled down hPD-1. FIG. 1A shows the presence of the overexpressed Flag-tagged PD-1 in the 293T cells co-transfected with a plasmid encoding a HA-tagged PD-1 (line 1), or cells transfected with HA-tagged PD-1 plasmid alone (line 2).

FIG. 1B shows the presence of overexpressed HA-tagged PD1. FIG. 1C shows that anti-Flag antibody pulled down Flag-tagged PD-1. FIG. 1D shows that Flag-tagged PD-1 pulled down the overexpressed HA-tagged PD-1. Without Flag-tagged PD-1, there was no HA-tagged PD-1 pulled down, indicating the PD-1 and PD-1 binding is specific.

FIG. 2 shows the results of pull-down assays using a fusion protein composed of a hPD-1 extracellular domain fused with the Fc portion of hIgG (hPD-1 Fc). The results show that PD-1 binding PD-1 is specific. As shown in the figure, a deglycosylated hPD-1 Fc and the positive control hPD-L1 Fc, but not hFc or hPD1 Fc, pulled down over-expressed HA tagged hPD-1.

FIG. 3A-FIG. 3B show the results of co-immunoprecipitation assay by which it was shown that non glycosylated PD-1 was specifically pulled down by PD-1. FIG. 3A shows the presence of the glycosylated and non-glycosylated Flag-tagged PD-1 in the 293T cells co-transfected with a plasmid encoding a Flag-tagged PD-1 and a plasmid encoding a HA-tagged PD1, with (line 2) or without Tunicamycin treatment (line 1). FIG. 3B shows that HA-tagged PD-1 pulled down the non-glycosylated but did not pull down the glycosylated Flag-tagged PD-1.

FIG. 4A-FIG. 4B show the results of ELISA showing the specific binding of PD-1 on PD-1. FIG. 4A showed that deglycosylation of PD-1 greatly enhances PD1 and PD1 interaction, while deglycosylation of PD-1 dramatically reduces its binding to PD-L1 (FIG. 4B).

FIG. 5 shows the development of a human/mouse cross reactive anti deglycosylated PD-1 antibody IMT200. ELISA was used to measure the affinity of IMT200 binding to human or mouse PD-1 produced in E. coli.

FIG. 6A-FIG. 6B show the results of binding property of IMT200 and other known PD-1 blockers on glycosylated hPD-1 produced in mammalian cells (FIG. 6A) and on non-glycosylated hPD-1 produced in E. coli (FIG. 6B). The results show that IMT200 binds to both glycosylated and non-glycosylated hPD-1, while two known anti PD-1 antibodies, Nivolumab and Pembrolizumab, bind to glycosylated PD-1 only.

FIG. 7 shows the result of epitope mapping of IMT200 on hPD-1. ELISA was used to show that IMT200 binding epitope on hPD-1 is TDKLAAFPED (SEQ ID NO: 9).

FIG. 8A-FIG. 8B show results of ELISA showing the blocking of anti-PD-1 mAb IMT200 on the binding of PD-1 to PD-1. FIG. 8A showed that the monoclonal antibody IMT200 blocked the interaction between deglycosylated PD-1 polypeptides. FIG. 8B showed that IMT200 was not able to block binding between PD-L1 and PD-1, but mAb EH12 was able to block the binding between PD-L1 and PD-1. This indicates that the binding sites on PD-1 for PD-L1 and PD-1 are different.

FIG. 9A-FIG. 9C show the results of PD-1 expression on macrophages. Flow cytometry was used to show that PD-1 expresses well on RAW mouse macrophages (FIG. 9A) and human M2 macrophages (FIG. 9C), and to a less extent on human M1 macrophages (FIG. 9B).

FIG. 10A-FIG. 10B show the results of functional assays in which the blocking antibody IMT200 in combination with PD-1 blocking antibodies reversed the suppression of PD-1 on T cell activation. FIG. 10A shows that when mouse RAW macrophages were mixed with mouse DO11.10 T cells and treated with the combination of mouse PD-1 blocking antibody 29F and IMT200, more IL-2 was produced as compared to their single usage. FIG. 10B shows the similar observation in which when human M1 macrophages from one donor were mixed with human T cells from another donor and treated with the combination of human PD-1 blocking antibody EH12 and IMT200, more IFNgamma was produced as compared to their single usage.

FIG. 11A-FIG. 11C show the results of an experimental CT26 colon tumor model showing the anti-tumor activity of PD-1 antibody IMT200. As compared to isotype or IMT200 or PD1 Ab single agent treated groups, the combination of IMT200 and 29F treated group showed reduction of tumor size. FIG. 11A depicts mean tumor volumes. FIG. 11B depicts individual tumor volumes. FIG. 11C depicts tumor growth inhibition (TGI) per animal and summarizes the frequency of complete responses (CR).

FIG. 12 illustrates an ELISA assessment of PD1-PD1 interaction blockade by PD1-degly binding antibodies. Percent of PD1-PD1 binding in the absence of antibody is shown.

FIG. 13A-FIG. 13E illustrate ELISA assessments of anti-PD1 antibody binding to peptide fragments of PD1. FIG. 13A: mab3; FIG. 13B: mab5; FIG. 13C: mab9; FIG. 13D: mab10; and FIG. 13E: mab12.

### DETAILED DESCRIPTION OF THE DISCLOSURE

Human cancers harbor numerous genetic and epigenetic alterations, generating neoantigens potentially recognizable by the immune system. Although endogenous immune response to cancer is observed in preclinical models, in patients the response is ineffective and established cancers are often viewed as “self” and tolerated by the immune system. In addition, tumors may exploit several distinct mechanisms to actively suppress the host immune response. Among these mechanisms, immune checkpoints, involving various negative regulators of the immune system that normally terminate immune responses to mitigate collateral tissue damage, can be used by tumors to evade immune destruction.

Human PD-1 is one of the immune checkpoint proteins expressed by activated T and B cells and mediate immunosuppression. PD-1 is a monomeric both in solution as well as on cell surface. Zhang et al. Immunity Vol. (2004) 20, 337-347. PD-1 is a member of the CD28 family of receptors, which include CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2). Interactions between these ligands and PD-1 have been show to downregulate T cell activation and cytokine secretion.

Although it has been reported that PD-1 has both glycosylated and non-glycosylated forms, published literature focus on the role of glycosylation in the PD-1 pathway; for example, glycosylation is reported to play critical roles in membrane protein functions. (Curr. Med. Chem. 13:1141) and that glycosylation of PD-L1 and PD 1 is important to stabilize interaction between PD-L1 and PD-1 for suppression of T cell activity. (Nat. Commun. 7:12632; WO 2017172617). Further, commercial anti PD-1 antibodies, such as Nivolumab and Pembrolizumab, are raised by immunizing animals with the glycosylated form of PD-1 polypeptide. To date, the roles of the non-glycosylated form in immunosuppression remain largely unknown.

The disclosure herein is based on the surprising discovery of the interaction between non-glycosylated PD-1 polypeptides that are located on the cell surface of different immune cells (e.g., as “trans interaction”) and this interaction contributes to suppression of T cell activation. In some embodiments, the disclosure provides methods that restore T cell activation by administering an inhibitor that can interfere with this interaction and optionally in combination with a glycosylated PD-1 inhibitor to activate immune response and treat tumor.

### Interaction Between Non-Glycosylated PD-1

Cancer cells in a solid tumor are able to form a tumor microenvironment in their surroundings to support the growth and metastasis of the cancer cells. A tumor microenvironment is the cellular environment in which the tumor exists, including surrounding blood vessels, immune cells, fibroblasts, other cells, soluble factors, signaling molecules, an extracellular matrix, and mechanical cues that can promote neoplastic transformation, support tumor growth and invasion, protect the tumor from host immunity, foster therapeutic resistance, and provide niches for dormant metastases to thrive. The tumor and its surrounding microenvironment are closely related and interact constantly. Tumors can influence their microenvironment by releasing extracellular signals, promoting tumor angiogenesis and inducing peripheral immune tolerance, while the immune cells in the microenvironment can affect the growth and evolution of cancerous cells. See Swarts et al. “Tumor Microenvironment Complexity: Emerging Roles in Cancer Therapy,” Cancer Res, vol., 72, pages 2473-2480, 2012.

Tumor-associated macrophages (TAMs) are a type of inflammatory cells that are recruited by tumor cells and infiltrate tumor tissues to the tumor microenvironment. TAMs, through interactions with tumor cells, can promote tumor development and progression by promoting angiogenesis, matrix remodeling and suppressing adaptive immunity. TAMs comprise M1 and M2 subtypes, which induce a Th1 and Th2 immune responses, respectively.

As used herein, and unless otherwise specified, the term “programmed death-1” or “PD-1” refers to PD-1 from any vertebrate source, including mammals such as primates (e.g., humans (NP_005009), cynomolgus monkey (ABR15751)), dogs (domestic dog XP_543338), and rodents (e.g., mice (CAA48113) and rats (NP_001100397)).

Unless otherwise specified, PD-1 also includes different modified forms of PD-1, including but not limited to phosphorylated PD-1 and unphosphorylated PD-1, glycosylated and non-glycosylated PD-1, and ubiquitinated and non-ubiquitnated PD-1, etc.

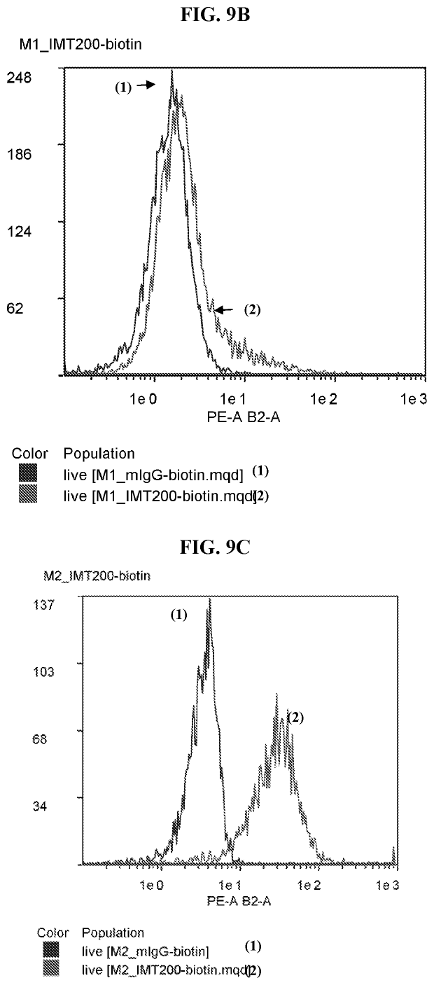

Glycosylation is a posttranslational modification that is initiated in the endoplasmic reticulum (ER) and subsequently processed in the Golgi (Schwarz & Aebi, Current Opinion in Structural Biology 21, 576-582(2011)). This type of modification is first catalyzed by a membrane-associated oligosaccharyl transferase (OST) complex that transfers a preformed glycan composed of oligosaccharides to an asparagine (Asn) side-chain acceptor located within the NXT motif (-Asn-X-Ser/Thr-) (Cheung and Reithmeier, Methods 41(4): 451-59 (2007); Helenius and Aebi, Science 291 (5512): 2364-69 (2001)). The addition or removal of saccharides from the preformed glycan is typically mediated by a group of glycotransferases and glycosidases, respectively, which tightly regulate the N-glycosylation cascade in a cell- and location-dependent manner. Many of the existing PD-1 inhibitors, for example, Nivolumab and Pembrolizumab, are glycosylated PD-1 inhibitors, in that they can bind to the glycosylated form of PD-1, but not the non-glycosylated form. see FIG. 6B. Some of these glycosylated PD-1 inhibitors block the interaction between PD-1 and its ligand, i.e., PD-L1 and/or PD-L2. Despite the success of these glycosylated PD-1 inhibitors in clinic, there remain a large groups of cancer patients who cannot benefit from these therapies, i.e., the treatment is unable to sufficiently activate T cells in these patients.

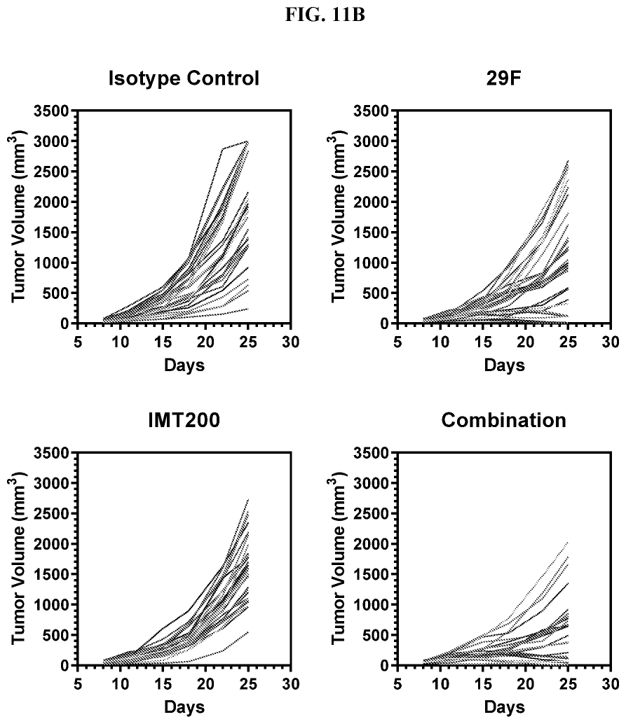

The inventors of this application have discovered that in addition to PD-L1 and PD-L2, a PD-1 polypeptide can also bind to another PD-1 polypeptide and the binding requires both PD-1 polypeptides to be non-glycosylated. The inventors of this application have found that non-glycosylated PD-1s are expressed in RAW macrophages, M2 macrophages, but not M1 macrophages. An illustrative example is shown in Example 4, which shows detection of high level expression of non-glycosylated PD-1 on RAW and M2 macrophages (FIGS. 9A and 9C, respectively), but little or no expression on M1 macrophages (FIG. 9B).

The inventors of this application have discovered surprisingly the interaction of non-glycosylated PD-1 polypeptides can suppress immune response. In some embodiments, this interaction is between non-glycosylated PD-1 on macrophages (e.g., RAW macrophages or M2 macrophages) and non-glycosylated PD-1 on T cells. This may contribute to the phenomenon that using glycosylated PD-1 inhibitors alone in some circumstances cannot mount immune response to a level that is sufficient to achieve clinically significant benefit. The non-glycosylated PD-1 inhibitors disclosed herein can inhibit the interaction between the non-glycosylated PD-1 polypeptides; and especially when used in combination with glycosylated PD-1 inhibitors, can activate immune response and/or reduce tumor load. In some embodiments, the non-glycosylated PD-1 inhibitor does not block binding of PD-1 to its ligand PD-L1. In some embodiments, the non-glycosylated PD-1 inhibitor does not block binding of PD-1 to its ligand PD-L2. In some embodiments, the non-glycosylated PD-1 inhibitor blocks neither the binding of PD-1 to PD-L1 nor the binding of PD-1 to PD-L2. The PD-L1 or PD-L2 polypeptides may be ones that are derived from human, mouse, primate etc. PD-L1 human (NP_054862), mouse (NP_068693), rat (NP_001178883), Cynomolgus monkey (XP_005581836). PD-L2 human (NP_079515), mouse (NP_067371), rat (NP_001101052), Cynomolgus monkey (NP_005581839).

In some instances, the effect of a single agent of the non-glycosylated PD-1 inhibitor on T cell activation may be moderate. In some cases, when combined with administration of a glycosylated PD-1 inhibitor, activation (e.g., synergistic activation) of T cells is achieved. In some cases, the non-glycosylated PD-1 inhibitor disclosed herein is used as a single agent or in combination with a glycosylated PD-1 inhibitor to treat cancers or other diseases that could benefit from activation of immune response.

### Non-Glycosylated PD-1 Inhibitor

The disclosure provides a pharmaceutical combination and methods to activate (e.g., synergistically activate) a patient's immune response by administering to the patient a non-glycosylated PD-1 inhibitor and optionally a glycosylated PD-1 inhibitor. A non-glycosylated PD-1 inhibitor can be any molecule that inhibits the interaction between non-glycosylated PD-1 polypeptides and said inhibition results in activation of T cells. The non-glycosylated PD-1 inhibitor can be a protein (e.g., an antibody) or a small molecule. The non-glycosylated PD-1 inhibitor can be a protein (e.g., an antibody). The non-glycosylated PD-1 inhibitor can be a small molecule. An antibody that is a non-glycosylated PD-1 inhibitor is referred to as non-glycosylated PD-1 inhibitor antibody in this disclosure.

In some embodiments, also disclosed herein is a pharmaceutical combination which comprises a non-glycosylated PD-1 inhibitor that impairs an interaction between two non-glycosylated PD-1 polypeptides; a glycosylated PD-1 inhibitor that impairs an interaction between a glycosylated PD-1 polypeptide and a programmed cell death ligand; and a pharmaceutically acceptable vehicle or excipient.

In some embodiments, further provided herein is a method of disrupting an interaction between two non-glycosylated PD-1 polypeptides, comprising: contacting a non-glycosylated PD-1 inhibitor to a plurality of cells comprising two cells each expressing a non-glycosylated PD-1 polypeptide, wherein the non-glycosylated PD-1 inhibitor impairs the interaction between the two non-glycosylated PD-1 polypeptides.

In some embodiments, additionally provided herein is a non-glycosylated PD-1 inhibitor for use in a method to activate immune response in a subject. In some instances, the method comprises administering to the subject a non-glycosylated PD-1 inhibitor to activate the immune response, wherein the non-glycosylated PD-1 inhibitor impairs an interaction between two non-glycosylated PD-1 polypeptides. In some instances, the method further comprises use of the non-glycosylated PD-1 inhibitor in combination with a glycosylated PD-1 inhibitor in a method to activate immune response in a subject. In some embodiments, the method comprises administering to the subject a non-glycosylated PD-1 inhibitor and a glycosylated PD-1 inhibitor to activate the immune response, wherein the non-glycosylated PD-1 inhibitor impairs an interaction between two non-glycosylated PD-1 polypeptides, and wherein the glycosylated PD-1 inhibitor impairs an interaction between a glycosylated PD-1 polypeptide and a programmed cell death ligand. In some embodiments, the non-glycosylated PD-1 inhibitor and the glycosylated PD-1 inhibitor synergistically activate immune response in a subject. In some embodiments, the patient hosts a tumor and the synergistic activation of immune response results in reduction in tumor load. In some cases, the programmed cell death ligand is PD-L1 or PD-L2.

In some embodiments, further provided herein is a method of reducing tumor cells within a tumor microenvironment (TME) in a subject, which comprises contacting a plurality of cells located within the TME with a non-glycosylated PD-1 inhibitor. In some instances, the method further comprises use of the non-glycosylated PD-1 inhibitor in combination with a glycosylated PD-1 inhibitor in a method to reduce tumor cells. In some cases, the method comprises contacting a plurality of cells located within the TME with a non-glycosylated PD-1 inhibitor and a glycosylated PD-1 inhibitor.

Accordingly, also provided herein are methods of selecting compounds that can block the interaction between non-glycosylated PD-1 polypeptides comprising (a) contacting a library of compounds with a first non-glycosylated PD-1 polypeptide, and a second non-glycosylated PD-1, wherein the first and the second non-glycosylated PD-1 are distinguishable, and (b) selecting one or more compounds from the library that are capable of blocking the binding between first non-glycosylated PD-1 and the second non-glycosylated PD-1. In some embodiments, the first and the second non-glycosylated PD-1 polypeptides are distinguishable by a label that is present in the first, or the second, but not both, non-glycosylated PD-1 polypeptide. In some embodiments, the method further comprises (c) contacting the one or more compounds selected from step (b) with a glycosylated PD-1 inhibitor, a mixture comprising T cells and antigen presenting cells and (d) identifying one or more compounds that is capable of stimulating the T cells when combined with the glycosylated PD-1 inhibitor.

In some instances, the non-glycosylated PD-1 inhibitor binds to a region of the non-glycosylated PD-1 polypeptide comprising SEQ ID NO: 9.

In some cases, the non-glycosylated PD-1 inhibitor does not impair an interaction between a PD-1 polypeptide and the programmed cell death ligand.

In some cases, the non-glycosylated PD-1 inhibitor further binds to a glycosylated PD-1 polypeptide.

In some cases, a binding affinity of the non-glycosylated PD-1 inhibitor to the glycosylated PD-1 polypeptide is equivalent to a binding affinity of a control to the glycosylated PD-1 polypeptide. In some cases, the control is Nivolumab or Pembrolizumab.

In some instances, the non-glycosylated PD-1 inhibitor binds to human non-glycosylated PD-1 polypeptide, mouse non-glycosylated PD-1 polypeptide, or a combination thereof.

In some instances, the tumor cells are reduced by at least 5%, 10%, 15%, 20%, 25%, 30%, 35%, 40%, 45%, 50%, 60%, 70%, 80%, or 90%.

In some cases, the cancer is a solid tumor. In some cases, the solid tumor is breast cancer, bile duct cancer, bladder cancer, colorectal cancer, gastric cancer, liver cancer, multiple myeloma, ovarian cancer, pancreatic cancer, prostate cancer, or thyroid cancer. In some cases, the solid tumor is a metastatic solid tumor. In other cases, the solid tumor is a relapsed or refractory solid tumor.

In some cases, the cancer is a hematologic malignancy. In some cases, the hematologic malignancy is a metastatic hematologic malignancy. In other cases, the hematologic malignancy is a relapsed or refractory hematologic malignancy.

In some instances, the non-glycosylated PD-1 inhibitor impairs an interaction between two non-glycosylated PD-1 polypeptides. In some cases, the interaction is impaired by about 20%, 30%, 40%, 50%, 60%, 70%, 80%, 90%, 95%, or 100%. In some cases, the interaction is impaired by about 1-fold, 2-fold, 3-fold, 4-fold, 5-fold, 10-fold, 20-fold, 30-fold, 40-fold, 50-fold, 100-fold, 200-fold, 500-fold, or more. In some cases, the non-glycosylated PD-1 inhibitor blocks the interaction between two non-glycosylated PD-1 polypeptides.

i. Non-Glycosylated PD-1 Inhibitor Antibodies

In some embodiments, the non-glycosylated PD-1 inhibitor is an antibody or its binding fragments thereof. In one embodiment, the method for treating cancer comprises administering a non-glycosylated PD-1 inhibitor antibody. Such an antibody can block the interaction between non-glycosylated PD-1 polypeptides, which contributes to the increased activation of T cells. In another embodiment, the method for treating cancer comprises administering a non-glycosylated PD-1 inhibitor antibody with a glycosylated PD-1 inhibitor antibody.

In some embodiments, disclosed herein is an antibody (or a non-glycosylated PD-1 inhibitor antibody) that impairs an interaction between two non-glycosylated PD-1 polypeptides, wherein the antibody comprises three variable heavy chain complementarity-determining regions (CDRs) and three variable light chain CDRs, and wherein the antibody specifically binds to a region of the non-glycosylated PD-1 polypeptide comprising SEQ ID NO: 9.

In some embodiments, also disclosed herein is an antibody (or a non-glycosylated PD-1 inhibitor antibody) that impairs an interaction between two non-glycosylated PD-1 polypeptides, wherein the antibody comprises three variable heavy chain complementarity-determining regions (CDRs) and three variable light chain CDRs, and wherein the three heavy chain CDRs comprise SEQ ID NOs: 11, 13, and 15, respectively. In some instances, the three light chain CDRs comprise SEQ ID NOs: 18, 20, and 22, respectively. In some instances, the antibody further comprises a heavy chain variable region (VH) comprising SEQ ID NO: 7. In some cases, the antibody further comprises a light chain variable region (VL) comprising SEQ ID NO: 8. In some cases, the antibody specifically binds to a region of the non-glycosylated PD-1 polypeptide comprising SEQ ID NO: 9.

In some embodiments, further disclosed herein are an antibody (or a non-glycosylated PD-1 inhibitor antibody) that comprises three heavy chain CDRs selected from the heavy chain CDRs of mab3, mab9, and mab10, and three light chain CDRs selected from the light chain CDRs of mab3, mab9, and mab10. In some instances, the antibody comprises a heavy chain variable region selected from the heavy chain CDRs of mab3, mab9, and mab10 and a light chain variable region selected from the heavy chain CDRs of mab3, mab9, and mab10. In some instances, the antibody specifically binds to a region of the non-glycosylated PD-1 polypeptide comprising SEQ ID NO: 9.

In some instances, the antibody is a humanized antibody or binding fragments thereof. In some instances, the antibody comprises a monoclonal antibody or binding fragments thereof. In some instances, the antibody comprises a monovalent Fab′, a divalent Fab2, a single-chain variable fragment (scFv), a diabody, a minibody, a nanobody, a single-domain antibody (sdAb), or a camelid antibody or binding fragment thereof. In some instances, the antibody comprises a bispecific antibody or its binding fragments thereof.

In some instances, the antibody is a full-length antibody, optionally comprising an Fc region selected from IgG1, IgG2, or IgG4. In some instances, the IgG2 is IgG2a or IgG2b.

In some instances, the antibody is an isolated antibody.

In some instances, the antibody (or a non-glycosylated PD-1 inhibitor antibody) is IMT200.

In some instances, the antibody (or a non-glycosylated PD-1 inhibitor antibody) is mab3 (clone name: 3E5). Hybridomas of antibody mab3 (clone: 3E5) is deposited with American Type Culture Collection (ATCC), Patent Depository, at 10801 University Boulevard, Manassas, Va. 20110, USA, on Apr. 4, 2019. The case number is: 55278-703.601.

In some instances, the antibody (or a non-glycosylated PD-1 inhibitor antibody) is mab9 (clone name: 5E9). Hybridomas of antibody mab9 (clone: 5E9) is deposited with American Type Culture Collection (ATCC), Patent Depository, at 10801 University Boulevard, Manassas, Va. 20110, USA, on Apr. 4, 2019. The case number is: 55278-703.601.

In some instances, the antibody (or a non-glycosylated PD-1 inhibitor antibody) is mab10 (clone name: 5G10). Hybridomas of antibody mab10 (clone: 5G10) is deposited with American Type Culture Collection (ATCC), Patent Depository, at 10801 University Boulevard, Manassas, Va. 20110, USA, on Apr. 4, 2019. The case number is: 55278-703.601.

In some cases, also described herein is a method of disrupting an interaction between two non-glycosylated PD-1 polypeptides, comprising: contacting a non-glycosylated PD-1 inhibitor antibody to a plurality of cells comprising two cells each expressing a non-glycosylated PD-1 polypeptide, wherein the non-glycosylated PD-1 inhibitor antibody impairs the interaction between the two non-glycosylated PD-1 polypeptides

In some instances, further described herein is a method of activating an immune response in a subject in need thereof, comprising: administering to the subject a non-glycosylated PD-1 inhibitor antibody and a glycosylated PD-1 inhibitor antibody to activate the immune response, wherein the non-glycosylated PD-1 inhibitor antibody impairs an interaction between two non-glycosylated PD-1 polypeptides, and wherein the glycosylated PD-1 inhibitor antibody impairs an interaction between a glycosylated PD-1 polypeptide and a programmed cell death ligand.

In some instances, additionally described herein is a method of reducing tumor cells within a tumor microenvironment (TME) in a subject, which comprises contacting a plurality of cells located within the TME with a non-glycosylated PD-1 inhibitor antibody and a glycosylated PD-1 inhibitor antibody.

In some cases, the non-glycosylated PD-1 inhibitor antibody does not impair an interaction between a PD-1 polypeptide and the programmed cell death ligand.

In some cases, the non-glycosylated PD-1 inhibitor antibody further binds to a glycosylated PD-1 polypeptide.

In some cases, a binding affinity of the non-glycosylated PD-1 inhibitor antibody to the glycosylated PD-1 polypeptide is equivalent to a binding affinity of a control to the glycosylated PD-1 polypeptide. In some cases, the control is Nivolumab or Pembrolizumab.

In some instances, the non-glycosylated PD-1 inhibitor antibody binds to human non-glycosylated PD-1 polypeptide, mouse non-glycosylated PD-1 polypeptide, or a combination thereof.

In some instances, the tumor cells are reduced by at least 5%, 10%, 15%, 20%, 25%, 30%, 35%, 40%, 45%, 50%, 60%, 70%, 80%, or 90% in the presence of a non-glycosylated PD-1 inhibitor antibody.

In some instances, the non-glycosylated PD-1 inhibitor antibody impairs an interaction between two non-glycosylated PD-1 polypeptides. In some cases, the interaction is impaired by at least or about 20%, 30%, 40%, 50%, 60%, 70%, 80%, 90%, 95%, or 100%. In some cases, the interaction is impaired by at least or about 50%, 60%, 70%, 80%, 90%, or more. In some cases, the interaction is impaired by at least or about 1-fold, 2-fold, 3-fold, 4-fold, 5-fold, 10-fold, 20-fold, 30-fold, 40-fold, 50-fold, 100-fold, 200-fold, 500-fold, or more. In some cases, the non-glycosylated PD-1 inhibitor antibody blocks the interaction between two non-glycosylated PD-1 polypeptides.

### Generating Non-Glycosylated PD-1 Inhibitor Antibodies

Non-glycosylated PD-1 inhibitor antibodies can be developed using methods well known in the art. See, for example, Kohler and Milstein, Nature 256: 495 (1975), and Coligan et al. (eds.), CURRENT PROTOCOLS IN IMMUNOLOGY, VOL. 1, pages 2.5.1-2.6.7 (John Wiley & Sons 1991). Non-glycosylated PD-1 antibodies can be generated by immunizing animals with non-glycosylated PD-1 polypeptide or an epitope thereof. In some cases, the non-glycosylated PD-1 or an epitope thereof is a recombinant polypeptide produced in an expression system in which no glycosylation occurs. Non-limiting examples of such expression system include, e.g., E. coli.

In some embodiments, the non-glycosylated PD-1 inhibitor antibody is generated by immunizing animals with a polypeptide comprising SEQ ID NO: 4. In some embodiments, the non-glycosylated PD-1 inhibitor antibody is generated by immunizing animals with a polypeptide comprising SEQ ID NO: 9.

Monoclonal antibodies can be obtained by injecting mice with a composition comprising an antigen, e.g. a non-glycosylated PD-1 or an epitope thereof, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

Monoclonal antibodies produced can be isolated and purified from hybridoma cultures by a variety of well-established techniques. Such isolation techniques include affinity chromatography with Protein-A Sepharose, size-exclusion chromatography, and ion-exchange chromatography. See, for example, Coligan at pages 2.7.1-2.7.12 and pages 2.9.1-2.9.3. Also, see Baines et al., “Purification of Immunoglobulin G (IgG),” in METHODS IN MOLECULAR BIOLOGY, VOL. 10, pages 79-104 (The Humana Press, Inc. 1992). After the initial raising of antibodies to the target protein, the antibodies can be sequenced and subsequently prepared by recombinant techniques. Humanization and chimerization of murine antibodies and antibody fragments are well known to those skilled in the art. See, for example, Leung et al. Hybridoma 13:469 (1994); US20140099254 A1.

Human antibodies can be produced using transgenic mice that have been genetically engineered to produce specific human antibodies in response to antigenic challenge using the target protein. See Green et al., Nature Genet. 7: 13 (1994), Lonberg et al., Nature 368:856 (1994). Human antibodies against the target protein can also be constructed by genetic or chromosomal transfection methods, phage display technology, or by in vitro activated B cells. See e.g., McCafferty et al., 1990, Nature 348: 552-553; U.S. Pat. Nos. 5,567,610 and 5, 229,275.

In some embodiments, the non-glycosylated PD-1 inhibitor is an antibody that is capable of binding to non-glycosylated PD-1 and interfering with the interaction between two or more non-glycosylated PD-1 polypeptides. In some cases, the method comprises administering to a patient hosting a tumor a non-glycosylated PD-1 inhibitor and a glycosylated PD-1 inhibitor in amounts that are effective to reduce the tumor load. In some cases, the combination of the two inhibitors can reduce tumor burden by at least 20%, e.g., at least 30%, at least 40%, or at least 46% in a mouse model over the treatment period, e.g., a period of three to twelve weeks. In some cases, the combination can activate immune response by at least 2 fold, at least 3 fold, at least 4 fold as measured by any of the assays known in the art, for example, the Mixed Lymphocyte Reaction (MLR) assay. For example, the activation of immune response can be detected by an increase in T cell proliferation, an increase in interferon-gamma production, and/or an increase in IL-2 secretion in an MLR assay.

In one embodiment, the non-glycosylated PD-1 inhibitor antibodies an antibody that recognizes one or more of the following polypeptides: human PD-1 (SEQ ID NO: 4), mouse PD-1 (SEQ ID NO: 2), and a PD-1 epitope comprising a polypeptide having the sequence set forth in SEQ ID NO: 9. In some embodiments, the non-glycosylated PD-1 is generated by immunizing mice with an epitope comprising a polypeptide having the sequence set forth in SEQ ID NO: 9.

### Modifying Non-Glycosylated PD-1 Inhibitor Antibodies

Non-glycosylated PD-1 inhibitor antibodies may also be produced by introducing conservative modifications relative to the existing non-glycosylated PD-1 inhibitor antibodies. For example, a modified non-glycosylated PD-1 inhibitor antibody may comprise heavy and light chain variable regions, and/or a Fc region that are homologous to the counterparts of an antibody produced above. The modified non-glycosylated PD-1 inhibitor antibody that can be used for the method disclosed herein must retain the desired functional properties of being able to block the interactions between non-glycosylated PD-1 polypeptides.

Non-glycosylated PD-1 inhibitor antibodies described herein can be linked to another functional molecule, e.g., another peptide or protein (albumin, another antibody, etc.), toxin, radioisotope, cytotoxic or cytostatic agents. For example, the antibodies can be linked by chemical cross-linking or by recombinant methods. The antibodies may also be linked to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192; or 4,179,337. The antibodies can be chemically modified by covalent conjugation to a polymer, for example, to increase their circulating half-life. Exemplary polymers and methods to attach them are also shown in U.S. Pat. Nos. 4,766,106; 4,179,337; 4,495,285; and 4,609,546.

Non-glycosylated PD-1 inhibitor antibodies may also be produced by altering protein modification sites. For example, sites of glycosylation of the antibody can be altered to produce an antibody lacking glycosylation and the so modified non-glycosylated PD-1 inhibitor antibodies typically have increased affinity of the antibody for antigen. Antibodies can also be pegylated by reacting with polyethylene glycol (PEG) under conditions in which one or more PEG groups become attached to the antibody. Pegylation can increase the biological half-life of the antibody. Antibodies having such modifications can also be used to treat the tumors so long as it retains the desired functional properties of blocking the interaction between non-glycosylated PD-1 polypeptides.

The antibodies may also be tagged with a detectable, or functional, label. Detectable labels include radiolabels such as ¹³¹I or ⁹⁹Tc, which may also be attached to antibodies using conventional chemistry. Detectable labels also include enzyme labels such as horseradish peroxidase or alkaline phosphatase. Detectable labels further include chemical moieties such as biotin, which may be detected via binding to a specific cognate detectable moiety, e.g., labeled avidin.

The antibodies may also comprise bispecific molecules comprising a non-glycosylated PD-1 inhibitor antibody, or a fragment thereof, of the invention. An antibody of the invention, or antigen-binding portions thereof, can be derivatized or linked to another functional molecule, e.g., another peptide or protein (e.g., another antibody or ligand for a receptor) to generate a bispecific molecule that binds to at least two different binding sites or target molecules. The antibody of the invention may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule of the invention, an antibody of the invention can be functionally linked (e.g., by chemical coupling, genetic fusion, noncovalent association or otherwise) to one or more other binding molecules, such as another antibody, antibody fragment, peptide or binding mimetic, such that a bispecific molecule results. In one illustrative embodiment, the bispecific antibody can be created using the knobs-into-holes strategy. The strategy typically involves first creating a first half of the antibody that recognizes a first antigen, e.g., non-glycosylated PD-1 polypeptide, and a second half of the antibody that recognizes a second antigen and then joining the two halves to create the bispecific antibody.

In some instances, the bispecific molecules comprises at least one first binding specificity for the non-glycosylated PD-1 polypeptide and a second binding specificity for a second target. In some embodiments, the second target is a known cancer target, for example, PD-L1. In some embodiments, the second target is an Fc receptor, e.g., human Fc.gamma.RI (CD64) or a human Fc.alpha. receptor (CD89). Therefore, the invention includes bispecific molecules capable of binding both to Fc.gamma.R or Fc.alpha.R expressing effector cells (e.g., monocytes, macrophages or polymorphonuclear cells (PMNs)), and to target cells expressing non-glycosylated PD-1 polypeptides, such as macrophages. These bispecific molecules target non-glycosylated PD-1 expressing cells to effector cell and trigger Fc receptor-mediated effector cell activities, such as phagocytosis of an PD-1 expressing cells, antibody dependent cell-mediated cytotoxicity (ADCC), cytokine release, or generation of superoxide anion.

In some instances, the antibodies comprise one or more mutations in the framework region, e.g., in the CH1 domain, CH2 domain, CH3 domain, hinge region, or a combination thereof. In some cases, the one or more mutations modulate Fc receptor interactions, e.g., to increase Fc effector functions such as ADCC and/or complement-dependent cytotoxicity (CDC). In some cases, the one or more mutations stabilize the antibody and/or increase the half-life of the antibody. In additional cases, the one or more mutations modulate glycosylation.

ii. Other Non-Glycosylated PD-1 Inhibitor Molecules

In another embodiment, the non-glycosylated PD-1 inhibitor disclosed herein is a small molecule, non-protein compound that interferes with the interaction between non-glycosylated PD-1 polypeptides and thus antagonizes a non-glycosylated PD-1's immune suppression function. These small molecules typically are organic molecules having a molecular weight between 50 daltons to 2500 daltons. The compounds can also be identified using any of the numerous approaches in combinatorial library methods known in the art and disclosed in, e.g., European patent application EP2360254. The combinatorial libraries include: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

iii. Non-Glycosylated PD-1 Inhibitor Conjugates

In some embodiments, the provided methods may include administering a non-glycosylated PD-1 inhibitor as described above that is conjugated to a therapeutic agent or an imaging agent. The therapeutic agent may be at least one of a cytotoxic agent, a chemotherapeutic agent, or an immunosuppressive agent. The linkage can be covalent or noncovalent (e.g., ionic). Where the non-glycosylated PD-1 inhibitor is an antibody or functional fragment thereof, such antibodies and functional fragments are referred to antibody-drug conjugates (ADC) or immunoconjugates. The antibody conjugates are useful for the local delivery of therapeutic agents, particularly cytotoxic or cytostatic agents, i.e. drugs to kill or inhibit tumor cells in the treatment of cancer allows targeted delivery of the drug moiety to tumors, and intracellular accumulation therein, where systemic administration of these unconjugated drug agents may result in unacceptable levels of toxicity to normal cells as well as the tumor cells sought to be eliminated. Therapeutic agents include but are not limited to toxins, including but not limited to plant and bacterial toxins, small molecules, peptides, polypeptides and proteins. Genetically engineered fusion proteins, in which genes encoding for an antibody, or fragments thereof including the Fv region, or peptides can be fused to the genes encoding a toxin to deliver a toxin to the target cell are also provided.

Techniques for conjugating such a therapeutic moiety to the non-glycosylated PD-1 inhibitor are well known, see, for example, Arnon et al., Monoclonal Antibodies And Cancer Therapy, Reisfeld et al. (eds.), pp. 243-56 (1985); Hellstrom et al., Controlled Drug Delivery (2nd Ed.), Robinson et al. (eds.), pp. 623-53 (1987); Thorpe, Monoclonal Antibodies '84:Biological And Clinical Applications, Pinchera et al. (eds.), pp. 475-506 (1985); “Analysis, Results, And Future Prospective Of The Therapeutic Use Of Radiolabeled Antibody In Cancer Therapy” In: Monoclonal Antibodies For Cancer Detection And Therapy, (Baldwin et al. eds.), pp. 303-316 (1985), and Thorpe et al., Immunol. Rev. 62:119-158 (1982). Alternatively, an antibody can be conjugated to a second antibody to form an antibody heteroconjugate as described in U.S. Pat. No. 4,676,980.

iv. Evaluating Candidate Non-Glycosylated PD-1 Inhibitors

A number of well-known assays can be used to assess whether a candidate, e.g., an antibody generated by immunizing an animal with an antigen comprising a non-glycosylated PD-1 protein or a test compound from combinatorial libraries, can block interaction between non-glycosylated PD-1 polypeptides. Non-limiting exemplary assays include one or more of the following: i) binding assays to test whether the candidate binds to the target protein, i.e., the non-glycosylated PD-1 polypeptide; ii) blocking assays to test whether the candidate can block the interaction between non-glycosylated PD-1 polypeptides; iii) cell-based functional assays to test whether the candidate, by blocking the interaction between the non-glycosylated PD-1 polypeptides, can activate T cells; and iv) in vivo efficacy assays to test whether the candidate, when administered to a subject in combination with a glycosylated PD-1 inhibitor, can reduce tumor load.

### Binding Assays

Any of the assays that are used to evaluate interaction of two molecules can be used to determine whether the candidate can bind to the target protein. Non-limiting exemplar assays include binding assays—such as Enzyme-Linked Immunosorbent Assays (ELISAs), radioimmunoassays (MA)—, Fluorescence-Activated Cell Sorting (FACS) analysis. In some cases, the target protein, i.e., the non-glycosylated PD-1 polypeptide, can be coupled with a radioisotope or enzymatic label such that binding of the target protein and the candidate can be determined by detecting the labeled target protein in a complex. For example, the target protein can be labeled with ¹²⁵I, ³⁵S, ¹⁴C, or ³H, either directly or indirectly, and the radioisotope detected by direct counting of radio-emission or by scintillation counting. Alternatively, the target protein molecules can be enzymatically labeled with, for example, horseradish peroxidase, alkaline phosphatase, or luciferase, and binding of the candidates to the target protein is determined by conversion of an appropriate substrate to product.

In some embodiments, immunoassays, such as Enzyme-linked immunosorbent assay (ELISA), can be used to evaluate a non-glycosylated PD-1 inhibitor candidate's binding specificity to its target protein. In some embodiments, samples comprising the candidate are added to the plates that are pre-coated with the target protein and incubated for a period of time. A labeled secondary antibody that recognizes the candidate can be added and signal from the labeled secondary antibody are detected. In some cases, the secondary antibody is conjugated to an enzyme and the binding can be assessed by addition of substrate specific for the enzyme and read at appropriate wavelength according to manufacturer's instructions. Non-limiting examples of enzymes that can be used include horseradish peroxidase and alkaline phosphatase. For horseradish peroxidase, the ABTS substrate can be used and readings at 415-490 nm can be taken to evaluate the capability of the candidate's binding to the non-glycosylated PD-1 polypeptide. Alternatively, the ELISA can also be performed by coating the candidate on the plate, adding the target protein to the plate and detecting the binding as described above.

The binding kinetics (e.g., binding affinity) of the candidates also can be assessed by standard assays known in the art, such as by Biacore analysis (Biacore AB, Uppsala, Sweden). In one exemplary assay, the target protein is covalently linked to a chip, e.g., a carboxy methyl dextran coated chip using standard amine coupling chemistry and kit provided by Biacore. Binding is measured by flowing the candidates in buffers (provided by Biacore AB) at appropriate concentrations a flow rate that is recommended by the manufacturer. The association kinetics and dissociate kinetics are recorded and the association kinetics and dissociate curves are fitted to a binding model using BIA evaluation software (Biacore AB). The K_{D}, Kₒₙ and K_{off} values of the interaction can be measured. Preferred non-glycosylated PD-1 polypeptide inhibitors can bind to their target protein with a Kd of 1×10⁻⁷ M or less, e.g., 5×10⁻⁷M or less or 1×10⁻⁸ M or less.

### Blocking Assays

Candidates that have demonstrated the ability to bind the target protein are then evaluated for their ability to block the interaction between non-glycosylated PD-1 polypeptides in a blocking assay. In some embodiments, the blocking assay is an immunoassay, e.g., an ELISA. In one embodiment, the method of determining if the candidate blocks the interaction between the non-glycosylated PD-1 polypeptides involves coating the plates with a first non-glycosylated PD-1, and adding a mixture of the candidate and a second non-glycosylated PD-1 polypeptide to the coated plates, and detecting the signal corresponding to the binding of the first and second non-glycosylated PD-1 polypeptide. The second non-glycosylated PD-1 polypeptide is typically distinguishable from the first non-glycosylated PD-1 polypeptide; for example, the second non-glycosylated PD-1 polypeptide can be conjugated to a detectable label such that the binding between the first and second non-glycosylated PD-1 polypeptides can be detected by the signal from the detectable label. A decrease in signal as compared to control reactions, in which no candidate is added, indicates the candidate is capable of the blocking the interaction between non-glycosylated PD-1 polypeptides. An exemplary blocking assay that can be used to determine whether a candidate can block the interaction between the non-glycosylated PD-1 polypeptides are described in Example 3.

In some embodiments, the blocking assay is a flow cytometry assay. In general, the candidate is mixed with a first non-glycosylated PD-1 polypeptide, and the mixture is added to cells overexpressing a second non-glycosylated PD-1. The binding of the non-glycosylated PD-1 polypeptide on the cell surface can be detected by fluorescently labeled antibodies. A decrease in signal in reactions containing the candidate as compared to control indicates that the candidate can block the interaction between non-glycosylated PD-1 polypeptides.

### Functional Assays

In some cases, candidates that have demonstrated binding to target proteins are further evaluated for its ability to increase activation of T cells when used in combination with a glycosylated PD-1 inhibitor using the Mixed Lymphocyte Reaction (MLR) assay. One exemplary assay is described in U.S. Pat. No. 8,008,449, the relevant disclosure is hereby incorporated by reference in its entirety. The MLR assay can be used to measure T cell proliferation, production of IL-2 and/or IFN-γ. In one exemplary assay, a candidate, at different concentrations, is added to purified T cells cultured with allogeneic antigen presenting cells (APCs), e.g., macrophages. A glycosylated PD-1 inhibitor is also added to the reaction. The cells are then cultured in the presence of the candidate for a period of between 4-7 days at 37° C. A certain volume of culture medium is then taken for cytokine measurement. The levels of IFN-gamma and other cytokines can be measured. Methods for measuring cytokine production are well known and commercial kits are readily available, e.g., OptEIA ELISA kits (BD Biosciences). In some embodiments, cells are cultured in the presence of ³H-thymidine for a period of between 12 to 24 hours, e.g., 18 hours, and analyzed for the amount of incorporation of ³H-thymidine in the cells, which is positively correlated to cell proliferation. Results showing that, as compared to control, the culture containing the candidate in combination with the glycosylated PD-1 inhibitor shows increased T cell proliferation, increased production of IL-2, and/or IFN-gamma, and the increases are larger than the sum of respective increases in the culture treated with glycosylated PD-1 inhibitor alone and the increase in the culture treated with the candidate alone, indicate the candidate and the glycosylated PD-1 inhibitor is effective in synergistically activating T cells. One exemplary assay of MLR that can be used for evaluating the candidate's capability in activating T cells is disclosed in Example 3.

In some embodiments, the combination of the non-glycosylated PD-1 inhibitor disclosed herein and a glycosylated PD-1 inhibitor results in a synergistic activation of immune response. In some cases, this synergistic activation is characterized by that proliferation of T cells is at least 20%, at least 30%, at least 50%, at least 100%, at least 200%, at least 300%, at least 400%, or at least 500% greater than the proliferation of T cells that have been contacted by either the non-glycosylated PD-1 inhibitor or the glycosylated PD-1 inhibitor alone. In some embodiments, the synergistic activation is characterized by that the amount of the cytokines produced, e.g., IL-2 and/or IFN-γ, from T cells that have been contacted with the combination of the non-glycosylated PD-1 inhibitor and glycosylated PD-1 inhibitor is at least 20%, at least 30%, at least 50%, at least 100%, at least 200%, at least 300%, at least 400%, or at least 500% greater than the amount of cytokines produced from T cells that have been contacted with either inhibitor alone.

### In Vivo Assays

In another embodiment, an in vivo assay is used to evaluate whether a candidate is effective in treating cancer. In vivo assays can be done in tumor models, such as mouse tumor models, according to well-established procedures. In brief, the animals, e.g., mice, are implanted subcutaneously with human tumor cell lines. When the tumors grow and reach a certain size, e.g., between 100 and 300 mm³, the candidate and a glycosylated PD-1 inhibitor are administered to the mice at a predetermined frequency at appropriate dosages. The candidate, in combination with a glycosylated PD-1 inhibitor, can be administered by a number of routes, such as intraperitoneal injection or intravenous injection. The animals are monitored once or twice weekly for tumor growth for period of time which usually lasts 4 to 12 weeks. The tumors are measured three dimensionally (height×width×length) and tumor volumes are calculated. Mice are typically euthanized at the end of the experiment, when the tumors reach tumor end point, e.g., 1500 mm³, or the mice show significant weight loss, e.g., greater than 15%, greater than 20%, or greater than 25% weight loss. A result showing that a slower tumor growth in the candidate treated group as compared to controls, or a longer mean time to reach the tumor end point volume is an indication that the candidate has activity in inhibiting cancer growth.

In some embodiments, the combination of the non-glycosylated PD-1 inhibitor disclosed herein and a glycosylated PD-1 inhibitor results in a reduction (e.g., a synergistic reduction) of tumor load. In some cases, this reduction (e.g., synergistic reduction) of tumor load is characterized by that the reduction in tumor volume or amount in a subject, e.g., a mouse, treated with the combination of the non-glycosylated PD-1 inhibitor disclosed herein and a glycosylated PD-1 inhibitor is at least 20%, at least 30%, at least 50%, at least 100%, at least 200%, at least 300%, at least 400%, or at least 500% greater than the reduction in tumor volume or amount in a subject treated with either non-glycosylated PD-1 inhibitor or glycosylated PD-1 inhibitor alone.

### Patient Population

Patients who may benefit from the combination therapy of a non-glycosylated PD-1 inhibitor and a glycosylated PD-1 inhibitor include those who may or may not respond to the glycosylated PD-1 inhibitor therapy alone. In some embodiments, a patient who receives the combination therapy disclosed herein is a patient who has previously failed a glycosylated PD-1 inhibitor therapy, e.g., the patient received the glycosylated PD-1 inhibitor therapy but did not exhibit clinically significant benefit. In some embodiments, the patient who receives the combination therapy of a glycosylated PD-1 inhibitor and a non-glycosylated PD-1 inhibitor is one who may also benefit from a glycosylated PD-1 inhibitor therapy alone. Treating these patients with the combination of a non-glycosylated PD-1 inhibitor and a glycosylated PD-1 inhibitor, each in effective amounts, can synergistically activate immune response and/or confer clinically significant benefit, such as reducing tumor load.

### Evaluate the Efficacy of the Non-Glycosylated PD-1 Inhibitor Therapy

The non-glycosylated PD-1 inhibitor therapy disclosed herein can reduce the tumor load and confer clinically significant benefit to cancer patients, especially when combined with a glycosylated PD-1 inhibitor. In some embodiments, the non-glycosylated PD-1 inhibitor is a humanized antibody. In some embodiments, the non-glycosylated PD-1 inhibitor is a human antibody. Methods for measuring these responses are well-known to skilled artisans in the field of cancer therapy, e.g., as described in the Response Evaluation Criteria in Solid Tumors (“RECIST”) guidelines.

In one approach, the tumor load is measured by assaying expression of tumor-specific biomarkers. This approach is especially useful for metastatic tumors. A tumor-specific biomarker is a protein or other molecule that is unique to cancer cells or is much more abundant in them as compared to non-cancer cells. Useful biomarkers for various cancers are known, Non-limiting examples of tumor-specific genetic markers include, alpha-fetoprotein (AFP) for liver cancer, beta-2-microglobulin (B2M) for multiple myeloma; beta-human chorionic gonadotropin (beta-hCG) for choriocarcinoma and germ cell tumors; CA19-9 for pancreatic cancer, gall bladder cancer, bile duct cancer, and gastric cancer; CA-125 and HE4 for ovarian cancer; carcinoembryonic antigen (CEA) for colorectal cancer; chromogranin A (CgA) for neuroendocrine tumor; fibrin/fibrinogen for bladder cancer; prostate-specific antigen (PSA) for prostate cancer; and thyroglobulin for thyroid cancer.

Methods of measuring the expression levels of a tumor-specific genetic marker are well known. In some embodiments, mRNA of the genetic marker is isolated from the blood sample or a tumor tissue and real-time reverse transcriptase-polymerase chain reaction (RT-PCR) is performed to quantify expression of the genetic marker. In some embodiments, western blots, immunohistochemistry, or flow cytometry analysis are performed to evaluate the protein expression of the tumor-specific genetic marker. Typically the levels of the tumor-specific genetic marker are measured in multiple samples taken over time of the therapy of the invention, and a decrease in levels correlates with a reduction in tumor load.

In another approach, the reduction of tumor load by the non-glycosylated PD-1 inhibitor therapy disclosed herein is shown by a reduction in tumor size or a reduction of amount of cancer in the body. Measuring tumor size is typically achieved by imaging-based techniques. For example, computed tomography (CT) scan can provide accurate and reliable anatomic information about not only tumor shrinkage or growth but also progression of disease by identifying either growth in existing lesions or the development of new lesions or tumor metastasis.

In yet another approach, a reduction of tumor load can be assessed by functional and metabolic imaging techniques. These techniques can provide earlier assessment of therapy response by observing alterations in perfusion, oxygenation and metabolism. For example, ¹⁸F-FDG PET uses radiolabeled glucose analogue molecules to assess tissue metabolism. Tumors typically have an elevated uptake of glucose, a change in value corresponding to a decrease in tumor tissue metabolism indicates a reduction in tumor load. Similar imaging techniques are disclosed in Kang et al., Korean J. Radiol. (2012) 13(4) 371-390.

A patient receiving the therapy disclosed herein may exhibit varying degrees of tumor load reduction. In some cases, a patient can exhibit a Complete Response (CR), also referred to as “no evidence of disease (NED)”. CR means all detectable tumor has disappeared as indicated by tests, physical exams and scans. In some cases, a patient receiving the combination therapy disclosed herein can experience a Partial Response (PR), which roughly corresponds to at least a 50% decrease in the total tumor volume but with evidence of some residual disease still remaining. In some cases the residual disease in a deep partial response may actually be dead tumor or scar so that a few patients classified as having a PR may actually have a CR. Also many patients who show shrinkage during treatment show further shrinkage with continued treatment and may achieve a CR. In some cases, a patient receiving the therapy can experience a Minor Response (MR), which roughtly means a small amount of shrinkage that is more than 25% of total tumor volume but less than the 50% that would make it a PR. In some cases, a patient receiving the therapy can exhibit Stable Disease (SD), which means the tumors stay roughly the same size, but can include either a small amount of growth (typically less than 20 or 25%) or a small amount of shrinkage (Anything less than a PR unless minor responses are broken out. If so, then SD is defined as typically less 25%).

Desired beneficial or desired clinical results from the therapy may also include e. g., reduced (i.e., slowing to some extent and/or stop) cancer cell infiltration into peripheral organs; inhibited (i.e., slowing to some extent and/or stop) tumor metastasis; increased response rates (RR); increased duration of response; relieved to some extent one or more of the symptoms associated with the cancer; decreased dose of other medications required to treat the disease; delayed progression of the disease; and/or prolonged survival of patients and/or improved quality of life. Methods for evaluating these effects are well known and/or disclosed in, e.g., cancerguide.org/endpoints.html and RECIST guidelines, supra.

In some cases, the administration of a non-glycosylated PD-1 inhibitor as disclosed herein may reduce tumor burden by at least 20%, at least 30%, at least 40%, or at least 46% within the treatment period.

In some embodiments, the combination of the non-glycosylated PD-1 inhibitor disclosed herein and a glycosylated PD-1 inhibitor results in a reduction of tumor load. In some embodiments, the combination of the non-glycosylated PD-1 inhibitor and a glycosylated PD-1 inhibitor results a synergistic reduction of tumor load. In some cases, this synergistic reduction of tumor load is characterized by that the reduction in tumor volume or amount in a subject treated with the combination of the non-glycosylated PD-1 inhibitor disclosed herein and a glycosylated PD-1 inhibitor is at least 20%, at least 30%, at least 50%, at least 100%, at least 200%, at least 300%, at least 400%, or at least 500% greater than the reduction in tumor volume or amount in a subject treated with either the non-glycosylated PD-1 inhibitor or the glycosylated PD-1 inhibitor alone.

### Combination Therapies

This disclosure provides a combination therapy comprising a non-glycosylated PD-1 inhibitor and a glycosylated PD-1 inhibitor to reduce the tumor load in the patient. By “combination therapy” or “in combination with”, it is not intended to imply that the therapeutic agents must be administered at the same time and/or formulated for delivery together, although these methods of delivery are within the scope described herein.

The non-glycosylated PD-1 inhibitor and the glycosylated PD-1 inhibitor can be administered following the same or different dosing regimen. In some embodiments, the non-glycosylated PD-1 inhibitor and glycosylated PD-1 inhibitor are administered sequentially in any order during the entire or portions of the treatment period. In some embodiments, the non-glycosylated PD-1 inhibitor and glycosylated PD-1 inhibitor is administered simultaneously or approximately simultaneously (e.g., within about 1, 5, 10, 15, 20, or 30 minutes of each other). Non-limiting examples of combination therapies are as follows, non-glycosylated PD-1 inhibitor is “A” and the glycosylated PD-1 inhibitor, is “B”:

A/B/AB/A/BB/B/AA/A/BA/B/BB/A/AA/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

In some instances, the glycosylated PD-1 inhibitor is Nivolumab (Opdivo) from Bristol-Myers Squibb or Pembrolizumab (Keytruda) from Merck. In some cases, the glycosylated PD-1 inhibitor is EH12 or 29F.

In some embodiments, the combination therapy administered to the patient further comprises a third anti-cancer agent. Administration of the third anti-cancer agents to a patient will follow general protocols for the administration of such compounds, taking into account the toxicity, if any, of the therapy.

i. Targeted Therapy

In some embodiments, the third anti-cancer agent is a targeted therapeutic agent, i.e., includes agent is against specific molecular or genetic targets, such as those associated with receptor tyrosine kinases.

ii. Chemotherapy and Radiotherapy

In some embodiments, the third anti-cancer agent is a chemotherapeutic agent. Suitable for use in combination with the non-glycosylated PD-1 inhibitor and a glycosylated PD-1 inhibitor of the invention include agents that have the property of killing cancer cells or inhibiting cancer cell growth. As compared to targeted therapies as described above, chemotherapies function in a non-specific manner, for example, inhibiting the process of cell division known as mitosis, and generally excludes agents that more selectively block extracellular growth signals (i.e. blockers of signal transduction). These agents include, but are not limited to anti-microtubule agents (e.g., taxanes and vinca alkaloids), topoisomerase inhibitors and antimetabolites (e.g., nucleoside analogs acting as such, for example, Gemcitabine), mitotic inhibitors, alkylating agents, antimetabolites, anti-tumor antibiotics, mitotic inhibitors, anthracyclines, intercalating agents, agents capable of interfering with a signal transduction pathway, agents that promote apoptosis, proteosome inhibitors, and alike.

Alkylating agents are most active in the resting phase of the cell. These types of drugs are cell-cycle non-specific. Exemplary alkylating agents that can be used in combination with the NON-GLYCOSYLATED PD-1 INHIBITOR of the invention include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Antitumor antibiotics are chemo agents obtained from natural products produced by species of the soil fungus Streptomyces. These drugs act during multiple phases of the cell cycle and are considered cell-cycle specific. There are several types of antitumor antibiotics, including but are not limited to Anthracyclines (e.g., Doxorubicin, Daunorubicin, Epirubicin, Mitoxantrone, and Idarubicin), Chromomycins (e.g., Dactinomycin and Plicamycin), Mitomycin and Bleomycin.

Antimetabolites are types of chemotherapy treatments that are cell-cycle specific. When the cells incorporate these antimetabolite substances into the cellular metabolism, they are unable to divide. These class of chemotherapy agents include folic acid antagonists such as Methotrexate; pyrimidine antagonists such as 5-Fluorouracil, Foxuridine, Cytarabine, Capecitabine, and Gemcitabine; purine antagonists such as 6-Mercaptopurine and 6-Thioguanine; Adenosine deaminase inhibitors such as Cladribine, Fludarabine, Nelarabine and Pentostatin.

Exemplary anthracyclines that can be used in combination with the non-glycosylated PD-1 inhibitor of the invention include, e.g., doxorubicin (Adriamycin® and Rubex®); Bleomycin (Lenoxane®); Daunorubicin (dauorubicin hydrochloride, daunomycin, and rubidomycin hydrochloride, Cerubidine®); Daunorubicin liposomal (daunorubicin citrate liposome, DaunoXome®); Mitoxantrone (DHAD, Novantrone®); Epirubicin (Ellence); Idarubicin (Idamycin®, Idamycin PFS®); Mitomycin C (Mutamycin®); Geldanamycin; Herbimycin; Ravidomycin; and Desacetylravidomycin.

Antimicrotubule agents include vinca alkaloids and taxanes. Exemplary vinca alkaloids that can be used in combination with the NON-GLYCOSYLATED PD-1 INHIBITOR of the invention include, but are not limited to, vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®). Exemplary taxanes that can be used in combination with the non-glycosylated PD-1 inhibitor of the invention include, but are not limited to paclitaxel and docetaxel. Non-limiting examples of paclitaxel agents include nanoparticle albumin-bound paclitaxel (ABRAXANE, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17:617-620).

Exemplary proteosome inhibitors that can be used in combination with the non-glycosylated PD-1 inhibitor of the invention, include, but are not limited to, Bortezomib (Velcade®); Carfilzomib (PX-171-007, (S)-4-Methyl-N-((S)-1-(((S)-4-methyl-1-((R)-2-methyloxiran-2-yl)-1-oxope-ntan-2-yl)amino)-1-oxo-3-phenylpropan-2-yl)-2-((S)-2-(2-morpholinoacetamid-o)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(-2R)-2-methyl-2-oxiranyl]-2-oxo-1-(phenylmethyl)ethyl]-L-serinamide (ONX-0912).

In some embodiments, the chemotherapeutic agent is selected from the group consisting of chlorambucil, cyclophosphamide, ifosfamide, melphalan, streptozocin, carmustine, lomustine, bendamustine, uramustine, estramustine, carmustine, nimustine, ranimustine, mannosulfan busulfan, dacarbazine, temozolomide, thiotepa, altretamine, 5-fluorouracil (5-FU), 6-mercaptopurine (6-MP), capecitabine, cytarabine, floxuridine, fludarabine, gemcitabine, hydroxyurea, methotrexate, pemetrexed, daunorubicin, doxorubicin, epirubicin, idarubicin, SN-38, ARC, NPC, campothecin, topotecan, 9-nitrocamptothecin, 9-aminocamptothecin, rubifen, gimatecan, diflomotecan, BN80927, DX-895 If, MAG-CPT, amsacrine, etoposide, etoposide phosphate, teniposide, doxorubicin, paclitaxel, docetaxel, gemcitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, gemcitabine, Irinotecan, albumin-bound paclitaxel, Oxaliplatin, Capecitabine, Cisplatin, docetaxel, irinotecan liposome, and etoposide, and combinations thereof.

In certain embodiments, the chemotherapeutic agent is administered at a dose and a schedule that may be guided by doses and schedules approved by the U.S. Food and Drug Administration (FDA) or other regulatory body, subject to empirical optimization.

In still further embodiments, more than one chemotherapeutic agent may be administered simultaneously, or sequentially in any order during the entire or portions of the treatment period. The two agents may be administered following the same or different dosing regimens.

Radiotherapy requires maximized exposure of the affected tissues while sparing normal surrounding tissues. Interstitial therapy, where needles containing a radioactive source are embedded in the tumor, has become a valuable new approach. In this way, large doses of radiation can be delivered locally while sparing the surrounding normal structures. Intraoperative radiotherapy, where the beam is placed directly onto the tumor during surgery while normal structures are moved safely away from the beam, is another specialized radiation technique. Again, this achieves effective irradiation of the tumor while limiting exposure to surrounding structures. Despite the obvious advantage of approaches predicated upon local control of the irradiation, patient survival rate is still very low.

iii. Others Therapies

The present methods involving an non-glycosylated PD-1 inhibitor and a glycosylated PD-1 inhibitor can be combined with other means of treatment such as surgery, radiation, and/or hormonal therapy. Hormonal therapies can inhibit growth-promoting signals coming from classic endocrine hormones, for example, primarily estrogens for breast cancer and androgens for prostate cancer.

### Pharmaceutical Compositions

The non-glycosylated PD-1 inhibitors disclosed herein are useful in the manufacture of a pharmaceutical composition or a medicament for treating inflammatory diseases as described above. Pharmaceutical compositions or medicaments for use in the present invention can be formulated by standard techniques using one or more physiologically acceptable carriers or excipients. Suitable pharmaceutical carriers are described herein and in, e.g., “Remington's Pharmaceutical Sciences” by E.W. Martin. A non-glycosylated PD-1 inhibitor and their physiologically acceptable salts and solvates can be formulated for administration by any suitable route, including, but not limited to, orally, topically, nasally, rectally, parenterally (e.g., intravenous, subcutaneous, intramuscular, intraarterial, intradermal, intraperitoneal, intravitreal, intracerebral, or intracerebroventricular), and combinations thereof. In some instances, the non-glycosylated PD-1 inhibitor is formulated for systemic administration. In some instances, the non-glycosylated PD-1 inhibitor is formulated for local administration. In some embodiments, the therapeutic agent is dissolved in a liquid, for example, water or saline.

For oral administration, a pharmaceutical composition or a medicament disclosed herein can take the form of, e.g., a tablet or a capsule prepared by conventional means. Preferred are tablets and gelatin capsules comprising the active ingredient(s), together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate, (b) lubricants, e.g., silica, anhydrous colloidal silica, talcum, stearic acid, its magnesium or calcium salt (e.g., magnesium stearate or calcium stearate), metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulfate, and/or (f) absorbents, colorants, flavors and sweeteners. In some embodiments, the tablet contains a mixture of hydroxypropyl methylcellulose, polyethyleneglycol 6000 and titanium dioxide. Tablets may be either film coated or enteric coated according to methods known in the art.

Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable carriers, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

For topical administration, the compositions can be in the form of emulsions, lotions, gels, creams, jellies, solutions, suspensions, ointments, and transdermal patches. For delivery by inhalation, the composition can be delivered as a dry powder or in liquid form via a nebulizer. For parenteral administration, the compositions can be in the form of sterile injectable solutions and sterile packaged powders. Preferably, injectable solutions are formulated at a pH of about 4.5 to about 7.5.

The compositions can also be provided in a lyophilized form. Such compositions may include a buffer, e.g., bicarbonate, for reconstitution prior to administration, or the buffer may be included in the lyophilized composition for reconstitution with, e.g., water. The lyophilized composition may further comprise a suitable vasoconstrictor, e.g., epinephrine. The lyophilized composition can be provided in a syringe, optionally packaged in combination with the buffer for reconstitution, such that the reconstituted composition can be immediately administered to a patient.

The compounds can be encapsulated in a controlled drug-delivery system such as a pressure controlled delivery capsule (see, e.g., Takaya et al., J. Control Rel., 50:111-122 (1998)), a colon targeted delivery system, a osmotic controlled drug delivery system, and the like. The pressure controlled delivery capsule can contain an ethylcellulose membrane. The colon target delivery system can contain a tablet core containing lactulose which is overcoated with an acid soluble material, e.g., Eudragit E®, and then overcoated with an enteric material, e.g., Eudragit L®. The osmotic controlled drug delivery system can be a single or more osmotic unit encapsulated with a hard gelatin capsule (e.g., capsule osmotic pump; commercially available from, e.g., Alzet, Cupertino, Calif.). Typically, the osmotic unit contains an osmotic push layer and a drug layer, both surrounded by a semipermeable membrane.

### Dosage

Pharmaceutical compositions or medicaments can be administered to a subject at a therapeutically effective dose to treat the cancers as described herein. In some embodiments, the pharmaceutical composition or medicament is administered to a subject in an amount sufficient to elicit an effective therapeutic response in the subject.

Dose administered will vary depending on a number of factors, including, but not limited to, the subject's body weight, age, individual condition, surface area or volume of the area to be treated, and/or on the form of administration. The size of the dose also will be determined by the existence, nature, and extent of any adverse effects that accompany the administration of a particular compound in a particular subject. Preferably, the smallest dose and concentration required to produce the desired result should be used. Dosage should be appropriately adjusted for children, the elderly, debilitated patients, and patients with cardiac and/or liver disease. Further guidance can be obtained from studies known in the art using experimental animal models for evaluating dosage.

Dosage regimens are adjusted to provide the optimum desired response, e.g., a therapeutic response or minimal adverse effects. For administration of a non-glycosylated PD-1 inhibitor antibody or a glycosylated PD-1 inhibitor antibody, the dosage ranges from about 0.0001 to about 100 mg/kg, usually from about 0.001 to about 20 mg/kg, or about 0.01 to about 40 mg/kg, and more usually from about 0.01 to about 10 mg/kg, of the subject's body weight. Preferably, the dosage is within the range of 0.1-10 mg/kg body weight. For example, dosages can be 0.1, 0.3, 1, 3, 5 or 10 mg/kg body weight, and more preferably, 0.3, 1, 3, or 10 mg/kg body weight.

The dosing schedule is typically designed to achieve exposures that result in sustained receptor occupancy (RO) based on typical pharmacokinetic properties of an Ab. An exemplary treatment regime entails administration once per week, once every two weeks, once every three weeks, once every four weeks, once a month, once every 3 months or once every three to 6 months. The dosage and scheduling may change during a course of treatment. For example, dosing schedule may comprise administering the Ab: (i) every two weeks in 6-week cycles; (ii) every four weeks for six dosages, then every three months; (iii) every three weeks; (iv) 3-10 mg/kg body weight once followed by 1 mg/kg body weight every 2-3 weeks. In some embodiments, a dosage regimen for a non-glycosylated PD-1 inhibitor or the glycosylated PD-1 inhibitor of the invention comprises 0.3-10 mg/kg body weight, preferably 3-10 mg/kg body weight, more preferably 3 mg/kg body weight via intravenous administration, with the Ab being given every 14 days in up to 6-week or 12-week cycles until complete response or confirmed progressive disease.

In some embodiments, the ratio of the amount of the non-glycosylated PD-1 inhibitor per dose to the amount of the glycosylated PD-1 inhibitor per dose ranges from 1:0.05 to 1:20, e.g., from 1:0.2 to 1:10, from 1:0.5 to 1:10, from 1:0.5 to 1:3, or about 1:1.

In some cases, two or more antibodies with different binding specificities are administered simultaneously, in which case the dosage of each Ab administered falls within the ranges indicated. Antibody is usually administered on multiple occasions. Intervals between single dosages can be, for example, weekly, every 2 weeks, every 3 weeks, monthly, every three months or yearly. Intervals can also be irregular as indicated by measuring blood levels of Ab to the target antigen in the patient. In some methods, dosage is adjusted to achieve a plasma Ab concentration of about 1-1000 mg/ml and in some methods about 25-300 mg/ml.

In some cases, the non-glycosylated PD-1 inhibitor or the glycosylated PD-1 inhibitor is a compound and may be administered for multiple days at the therapeutically effective daily dose and the treatment may continue for a period ranging from three days to two weeks or longer. While consecutive daily doses are a preferred route to achieve a therapeutically effective dose, a therapeutically beneficial effect can be achieved even if the agents are not administered daily, so long as the administration is repeated frequently enough to maintain a therapeutically effective concentration of the agents in the subject. For example, one can administer the agents every day, every other day, or, if higher dose ranges are employed and tolerated by the subject, twice a week.

In some embodiments, the disclosure provides a unit dosage for oral administration to an individual of about 50 to 70 kg may contain between about 20 and 300 mg of the active ingredient. Typically, a dosage of the non-glycosylated PD-1 inhibitor is a dosage that is sufficient to achieve the desired effect. Optimal dosing schedules can be calculated from measurements of agent accumulation in the body of a subject. In general, dosage may be given once or more daily, weekly, or monthly. Persons of ordinary skill in the art can easily determine optimum dosages, dosing methodologies, and repetition rates.

Thus, in some embodiments, the pharmaceutical composition provided herein is a sterile solution comprising an antibody that is able to interfere with the interaction between the non-glycosylated PD-1 polypeptides on cell surface of immune cells in a tumor microenvironment, e.g., the solution comprising 10 μg-100 mg, e.g., 10 μg-40 mg, 100 μg-40 mg, or 1 mg-10 mg of antibody per kilogram of patient body weight in a solution of 100 ml suitable for intravenous delivery over a time period, e.g., 1-4 hour period. The antibody in the sterile solution can be a non-glycosylated PD-1 inhibitor antibody. In some embodiments, the antibody is a human antibody. In some embodiments, the antibody is a humanized antibody. In some embodiments, the antibody is a chimeric antibody. In some embodiments, the sterile solution further comprises one or more glycosylated PD-1 inhibitor antibodies. In some embodiments, the sterile solution further comprises one or more the targeted therapy agents, e.g., antibodies targeting receptor kinases and antibodies targeting angiogenesis pathway components. In some embodiments, the sterile solution further comprises one or more nanoparticles having a diameter between 10 and 100 nm, e.g., between 40 and 100 nm, or between 50 and 80 nm.

In some embodiments, the compositions of the invention are administered for one or more weeks, e.g., 1, 2, 3, 4, 5, 6, 7, 8, 9, 10, 11, 12, 13, 14, or more weeks. In yet other embodiments, the compounds are administered for one or more months, e.g., 1, 2, 3, 4, 5, 6, 7, 8, 9, 10, 11, 12, or more months.

Alternatively, the Ab can be administered as a sustained release formulation, in which case less frequent administration is required. Dosage and frequency vary depending on the half-life of the Ab in the patient. In general, human Abs shows the longest half-life, followed by humanized Abs, chimeric Abs, and nonhuman Abs. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

The dosage of a composition of the present invention can be monitored and adjusted throughout treatment, depending on severity of symptoms, frequency of recurrence, and/or the physiological response to the therapeutic regimen. Those of skill in the art commonly engage in such adjustments in therapeutic regimens.

### Certain Terminologies

Unless defined otherwise, all technical and scientific terms used herein have the same meaning as is commonly understood by one of skill in the art to which the claimed subject matter belongs. It is to be understood that the detailed description are exemplary and explanatory only and are not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

The terms “subject”, “patient” or “individual” are used herein interchangeably to refer to a human or animal. For example, the animal subject may be a mammal, a primate (e.g., a monkey), a livestock animal (e.g., a horse, a cow, a sheep, a pig, or a goat), a companion animal (e.g., a dog, a cat), a laboratory test animal (e.g., a mouse, a rat, a guinea pig, a bird), an animal of veterinary significance, or an animal of economic significance. None of the terms require or are limited to situations characterized by the supervision (e.g. constant or intermittent) of a health care worker (e.g. a doctor, a registered nurse, a nurse practitioner, a physician's assistant, an orderly or a hospice worker).

The terms “polypeptide,” “peptide,” and “protein” are used interchangeably herein to include a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” includes naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs include compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” include chemical compounds that have a structure that is different from the general chemical structure of an amino acid, but that functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

The term “synergistic” or “synergy” interchangeably refers to the interaction of two or more agents so that their combined effect is greater than the sum of their individual effects. Synergistic drug interactions can be determined using the median effect principle (see, Chou and Talalay (1984) Adv Enzyme Regul 22:27 and Synergism and Antagonism in Chemotherapy, Chou and Rideout, eds., 1996, Academic, pp. 61-102) and quantitatively determined by combination indices using the computer program Calcusyn (Chou and Hayball, 1996, Biosoft, Cambridge, Mass.). See also, Reynolds and Maurer, Chapter 14 in Methods in Molecular in Medicine, vol. 110: Chemosensitivity, Vol. 1: In vitro Assays, Blumenthal, ed., 2005, Humana Press. Combination indices (CI) quantify synergy, summation and antagonism as follows: CI<1 (synergy); CI=1 (summation); CI>1 (antagonism). A CI value of 0.7-0.9 indicates moderate to slight synergism. A CI value of 0.3-0.7 indicates synergism. ACI value of 0.1-0.3 indicates strong synergism. A CI value of <0.1 indicates very strong synergism.

The term “synergistically activating immune response” refers to the effect of activating immune response of two agents is greater than the sum of their individual effects of activating immune response. The activation of immune response can be measured by methods well known in the art, for example measuring T cell proliferation and/or production of IL-2 and/or IFN-γ in a Mixed Lymphocyte Reaction (MLR) assay.

The term “synergistically reducing tumor load” refers to the effect of reducing tumor load of two agents is greater than the sum of their individual effects of reducing tumor load. Tumor load can be measured by methods well-known in the art or disclosed below.

The term “therapeutically effective amount” or “effective mount” includes an amount or quantity effective, at dosages and for periods of time necessary, to achieve the desired therapeutic or prophylactic result. In the case where a desired result is achieved when a combination of two agents is used, the amount of each agent is an example of an effective amount for that agent.

The term “administering” includes oral administration, topical contact, administration as a suppository, intravenous, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. One skilled in the art will know of additional methods for administering a therapeutically effective amount of the non-glycosylated PD-1 inhibitor described herein to interfere with the interaction between non-glycosylated PD-1 polypeptides to decrease the caner load of a patient. By “co-administer” it is meant that a first compound described herein is administered at the same time, just prior to, or just after the administration of a second compound described herein.

The term “tumor” refers to an abnormal growth of tissue that results from excessive cell division. A tumor disclosed herein can be a malignant tumor or a benign tumor. A tumor can also be a solid tumor or a liquid tumor, such as leukemia.

The term “cancer” refers to a malignant tumor.

The term “tumor microenvironment” or “cancer microenvironment” refers to a cellular environment in which the tumor or cancer exists, including surrounding blood vessels, immune cells, fibroblasts, bone marrow-derived inflammatory cells, lymphocytes, signaling molecules and the extracellular matrix.

The term “immune response” refers to T cell-mediated and/or B cell-mediated immune responses. Exemplary immune responses include B cell responses (e.g., antibody production) T cell responses (e.g., cytokine production, and cellular cytotoxicity) and activation of cytokine responsive cells, e.g., macrophages. The term “activating immune response” refers to enhancing the level of T-cell-mediated and/or B cell-mediated immune response, using methods known to one of skilled in the art. In one embodiment, the level of enhancement is at least 20 50%, alternatively at least 60%, at least 70%, at least 80%, at least 90%, at least 100%, at least 120%, at least 150%, or at least 200%.

The term “recognizes” refers to a phenomenon that a molecule is able to specifically and selectively bind to a second molecule. Typically, a specific or selective binding will be at least twice background signal or noise and more typically more than 10 to 100 times background.

The term “non-glycosylated PD-1 inhibitor” refers to a molecule that inhibits the interaction between non-glycosylated PD-1 polypeptides that are located on different immune cells and the inhibition relieves T cell suppression. In some embodiments, the non-glycosylated PD-1 inhibitor does not block the interaction between PD-1 and its ligand PD-L1 and/or PD-L2. In some embodiments, the non-glycosylated inhibitor inhibits the interaction between non-glycosylated polypeptides on tumors and immune cells of different types.

The term “glycosylated PD-1 inhibitor” refers to a molecule that binds to the glycosylated PD-1, but does not bind to non-glycosylated PD-1. In some embodiments, the glycosylated PD-1 inhibitor inhibits the interaction between the PD-1 and PD-L1. Non-limiting examples of glycosylated PD-1 inhibitors include Nivolumab and Pembrolizumab.

The term “anti-glycosylated PD-1 therapy” refers to therapeutics using one or more glycosylated PD-1 inhibitors.

The term “non-glycosylated PD-1”, used interchangeably with “deglycosylated PD-1” refers to a PD-1 polypeptide that is not glycosylated.

The term “activating T cells” refers to the phenomenon that T cells are activated and engaged in signaling pathways that promote immune responses. The activation of T cells is typically accompanied with T cell proliferation and/or release of cytokines, e.g., interferon-gamma, IL-2, IL-5, IL-10, IL-12, or transforming growth factor (TGF)-beta.

The term “allogeneic” refers to denoting, relating to, or involving tissues or cells that are genetically dissimilar and hence immunologically incompatible, although from individuals of the same species.

The term “tumor load,” or “tumor burden” generally refers to the number of tumor cells, the size of a tumor, or the amount of tumor in the body in a subject at any given time. Tumor load can be detected by e.g., measuring the expression of tumor specific genetic markers and measuring tumor size by a number of well-known, biochemical or imaging methods disclosed herein, infra.

The term “antibody” is used in the broadest sense and specifically covers monoclonal antibodies, polyclonal antibodies, multi-specific antibodies, e.g., bispecific antibodies, chimeric antibodies, humanized antibodies, fully synthetic antibodies and antibody fragments so long as they exhibit the desired biologic activity, i.e., binding specificity. An antibody is a monomeric or multimeric protein comprising one or more polypeptide chains. An antibody binds specifically to an antigen and can be able to modulate the biological activity of the antigen. The term “antibody” also includes antibody fragments. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al., 1989, Nature 341:544-546) which consists of a single variable, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883), (viii) bispecific single chain Fv dimers (PCT/US92/09965) and (ix) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448). In certain embodiments, antibodies are produced by recombinant DNA techniques. Other examples of antibody formats and architectures are described in Holliger & Hudson, 2006, Nature Biotechnology 23(9):1126-1136, and Carter 2006, Nature Reviews Immunology 6:343-357 and references cited therein, all expressly incorporated by reference. In additional embodiments, antibodies are produced by enzymatic or chemical cleavage of naturally occurring antibodies.

The term “humanized antibody” refers to antibodies in which CDR sequences derived from the germline of another mammalian species, such as a mouse, have been grafted onto human framework sequences. The term “framework” refers to variable domain residues other than hypervariable region residues. The framework of a variable domain generally consists of four FR domains: FR1, FR2, FR3, and FR4. Framework region modifications may be made within the human framework sequences.

The term “human antibody” refers to an antibody that possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “chimeric antibody” refers to antibodies in which the variable region sequences are derived from one species and the constant region sequences are derived from another species, such as an antibody in which the variable region sequences are derived from a mouse antibody and the constant region sequences are derived from a human antibody.

The term “a checkpoint inhibitor therapy” refers to a therapy that suppresses a checkpoint pathway. Non-limiting examples of checkpoint inhibitor therapies include therapies that inhibit the PD1 signaling pathway and therapies that inhibit the CTLA4 signaling pathway. A checkpoint inhibitor therapy can be a peptide, an antibody, a nucleoside analog (e.g., an aptamer), a small molecule compound, or combinations thereof.

The term “allogeneic” refers to that the cells are obtained from individuals belonging to the same species but are genetically dissimilar.

The term “non-checkpoint inhibitor therapy” refers to a therapy that treats cancer through targeting pathways that are do not involve checkpoint inhibitors.

The term “primary tumor” refers to a tumor that is at a location of the body or a tissue where the particular cancer starts. Primary cancer is often referred to as the first or original cancer. Primary cancer is the opposite of metastasis, which refers to the migration of cancer cells from the original tumor site to produce cancer in other tissues.

The term “metastatic tumor” refers to a cancer that has spread from the site of origin (where it started) into different area(s) of the body.

The term a cancer is “suitable for treatment of a combination therapy of a non-glycosylated PD-1 inhibitor and a glycosylated PD-1 inhibitor” refers to a tumor that is likely to respond to treatment with a combination therapy of a non-glycosylated PD-1 inhibitor and a glycosylated PD-1 inhibitor, for example, the patient receiving the combination therapy is likely to have a beneficial clinical outcome, such as, overall survival rate, time to progression, disease-free survival, progression-free survival, tumor load reduction, or any of other beneficial clinical outcome as disclosed below or those according to the RECIST criteria.

### EXAMPLES

These examples are provided for illustrative purposes only and not to limit the scope of the claims provided herein.

### Example 1. PD-1 Specifically Binds to PD-1

This example describes various assays that have been conducted to evaluate the interaction between non-glycosylated PD-1 polypeptides. In Examples 1-6 of this application, unless otherwise stated, the non-glycosylated proteins were produced in E. coli.

### Binding Assays—Co-Immunoprecipitation

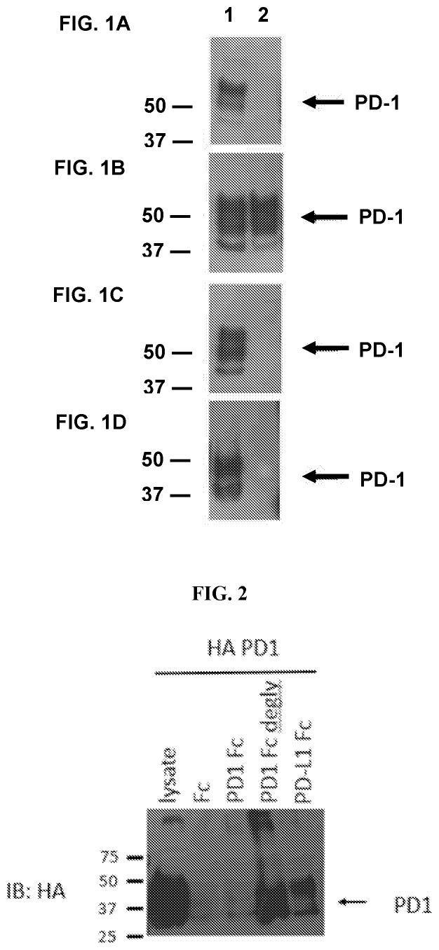

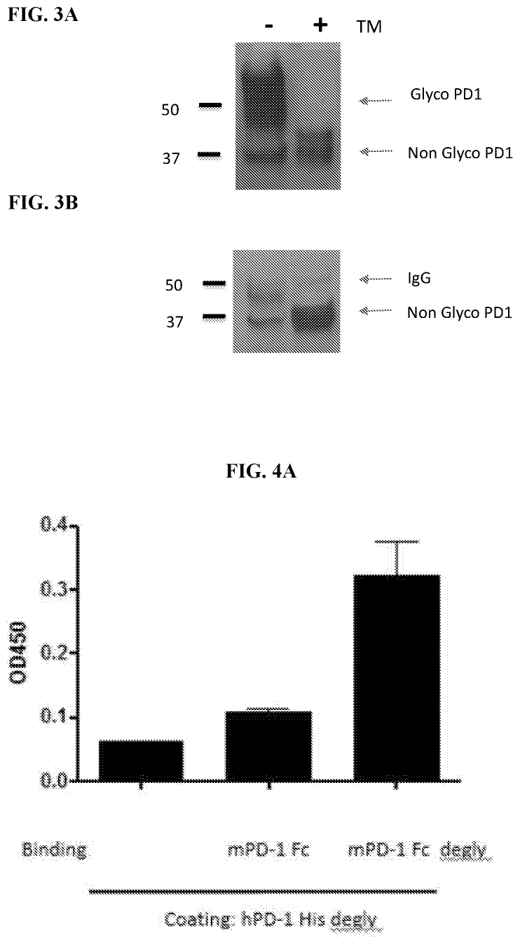

Co-immunoprecipitation experiments were performed to test whether PD-1 specifically interacts with PD-1. 293T cells were co-transfected with a plasmid encoding HA-tagged PD-1 with or without a plasmid encoding Flag-tagged PD-1. The transfection was performed using lipofectamine 3000 (Life Technologies) following manufacturer's protocols. The transfected cells were grown over night and then washed and lysed in 1 ml lysis buffer. The lysed cells were centrifuged and supernatant (the lysate) was collected. The lysates were prepared and separated on SDS PAGE and probed with anti-Flag (FIG. 1A) and anti-HA antibodies (FIG. 1B), respectively. Both the anti-Flag and the anti-HA antibodies were purchased from Sigma. The arrows in FIGS. 1A and 1B indicate the presence of PD-1.

For immunoprecipitation, anti-Flag antibody and protein G beads (Santa Cruz biotech) were added to the supernatant (the lysate) produced above. The beads and the lysates were incubated by rotating at 4° C. overnight to allow the Flag-tagged proteins to attach. The beads were then washed 3× with lysis buffer and mixed with 1×SDS PAGE sample buffer, boiled and separated on SDS-PAGE. The SDS-PAGE gel was transferred onto a membrane which was probed with anti-Flag antibody (FIG. 1C), or with anti-HA antibody (FIG. 1D).

The results, as shown in FIG. 1A-FIG. 1D, indicate that human PD-1 specifically pulled down human PD-1.

Additional co-immunoprecipitation experiments were performed to test if PD-1 specifically interacts with PD-1. HA tagged-human PD-1 plasmid was transfected into 293T cells, which were at 80% confluency. The transfections were performed in 10 cm plates using lipofectamine 3000 as described above. After overnight transfection, the cells were replaced on 10 cm plates that had been coated with 10 μg/ml human Fc, human PD1-Fc, deglycosylated human PD-1 Fc, or human PD-L1 Fc (R&D Systems) for 3 hours.

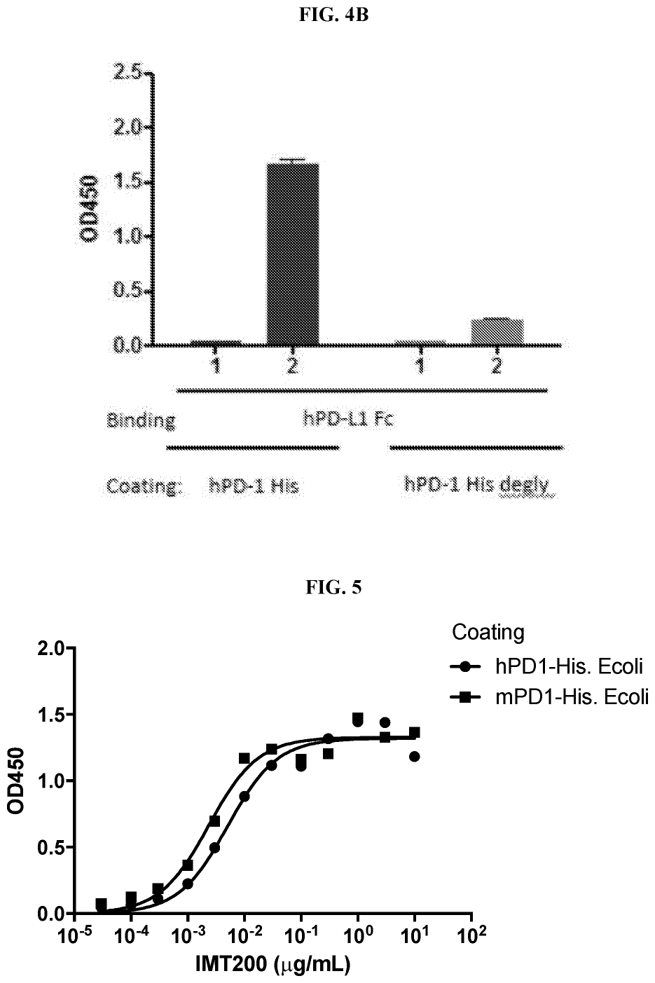

Deglycosylated human PD-1 Fc used in this experiment was prepared by treating human PD-1 Fc produced in a mammalian system with a deglycosylation kit, the Protein Deglycosylation Mix II (New England Biolabs, P6044). The reaction was then incubated at 37° C. overnight. The cells were washed once in 1×PBS, and then lysed in 1 ml lysis buffer. Cell lysates were collected and centrifuged. 30 μl of protein G beads was added to the supernatant formed after the centrifugation and incubated by rotating at 4° C. for overnight. The beads were then washed 3× with lysis buffer, followed by addition of 50 μl 1×SDS PAGE sample buffer. The samples containing the beads were boiled and separated on SDS-PAGE, transferred onto membrane. The membrane was then probed with ant-HA antibodies. As shown in FIG. 2, deglycosylated human PD-1 Fc specifically pulled down HA-tagged PD-1. In contrast, neither human Fc nor human PD1 Fc was able to pull down PD-1. This shows that PD-1 does not bind to Fc or PD1 Fc and that the binding between deglycosylated PD-1 and PD-1 is specific. As a positive control, hPD-L1 Fc also pulled down HA-tagged PD-1.

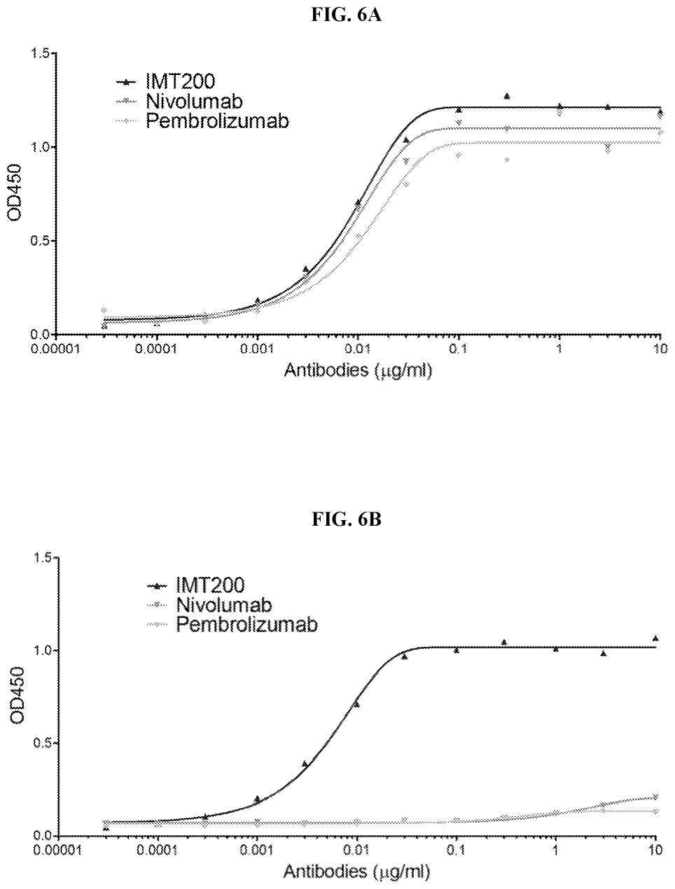

Another co-immunoprecipitation was carried out to confirm that only non-glycosylated PD1 polypeptides can interact with each other. 293T cells co-transfected with a plasmid encoding HA-tagged PD-1 and a plasmid encoding Flag-tagged PD-1 were treated with the protein glycosylation inhibitor tunicamycin (TM) overnight. The cells were then washed and lysed in 1 ml lysis buffer. The lysed cells were centrifuged and supernatant (the lysate) was collected. The lysates were prepared and separated on SDS PAGE and probed with anti-Flag (FIG. 3A). The arrows in FIG. 3A indicate the presence of glycosylated PD-1 and non-glycosylated PD-1.

For immunoprecipitation, anti-HA antibody and protein G beads were added to the supernatant (the lysate) produced above. The beads and the lysates were incubated by rotating at 4° C. overnight to allow the HA-tagged proteins to attach. The beads were then washed 3× with lysis buffer and mixed with 1×SDS PAGE sample buffer, boiled and separated on SDS-PAGE. The SDS-PAGE gel was transferred onto a membrane which was probed with ant-Flag antibody (FIG. 3B).

The results, as shown in FIG. 3B, indicate that only non-glycosylated PD-1 was specifically pulled down, confirming that the binding between PD1 and PD1 is sugar independent.

### Binding Assay—ELISA

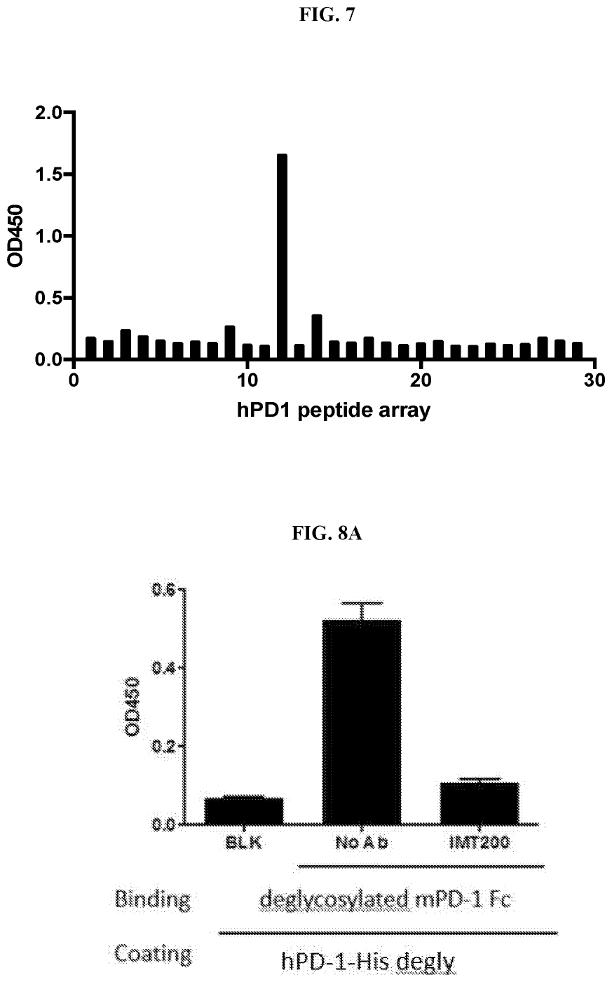

The interaction between the non-glycosylated PD-1 polypeptides was tested by ELISA. 96 well ELISA plates (ThermoFisher Scientific) were coated with hPD-1 His or deglycosylated hPD-1 His protein (R&D systems) in PBS and incubated at 4° C. overnight. The plates were washed three times with TBST and then blocked with PBS buffer containing 2% BSA at room temperature for 1 hour. Deglycosylated mPD-1 Fc (FIG. 4A) or hPD-L1 Fc (FIG. 4B) was added to the plates and incubated for one hour. Plates were then washed for three times and followed by incubation with anti-human-IgG-HRP (Jackson Immuno Research) for 1 h at room temperature. The plates were washed and a TMB substrate (GeneTex) was added until a color was developed. The reaction was then terminated with 1N HCl. The optical density (OD) was read at 450 nm. The results were expressed as the average OD of duplicates ±SD. The results showed that a non-glycosylated PD-1 can interact with another non-glycosylated PD-1 polypeptide (FIG. 4A), but non-glycosylated PD-1 does not interact with PD-L1 (FIG. 4B). This indicates that interaction between PD-1 and PD-L1 requires glycosylation of PD-1 polypeptides, while interaction between two PD-1 polypeptides requires deglycosylation of both PD-1 polypeptides (FIG. 4A).

### Example 2 Generation of Blocking Monoclonal Antibody (IMT200)

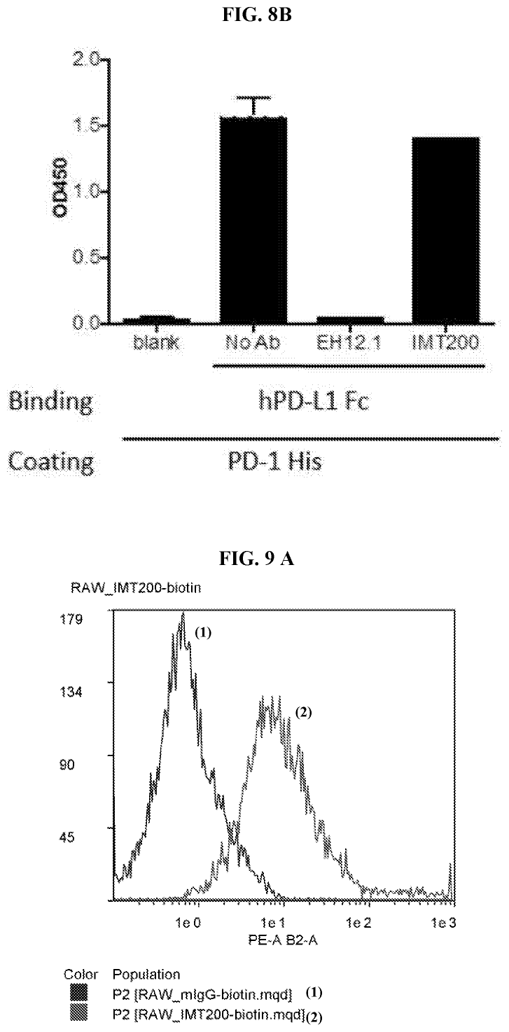

A panel of monoclonal antibodies against non-glycosylated, His-tagged, hPD-1 protein (BioVision) was generated and assayed for their activity in blocking PD-1 and PD-1 interaction. One monoclonal antibody, IMT200, was therefore identified and characterized as follows. First, ELISA was used to measure the affinity of IMT200 binding to human or mouse PD-1 (FIG. 5). Briefly, non-glycosylated, His-tagged, hPD-1 protein produced in E. coli, or His-tagged, non-glycosylated mPD-1 (ProSci) at 0.1 ug/ml was coated in 96-well plate for overnight. After three times wash, plates were blocked with PBS buffer containing 2% BSA at room temperature for 1 hour. IMT200 at 3× serial dilution starting from 10 ug/ml was added to the plates and incubated for one hour. Plates were then washed for three times and followed by incubation with anti-mouse IgG-HRP (Jackson Immuno Research) for 1 h at room temperature. The color was developed as stated above. The results showed that the affinities of IMT200 binding to non-glycosylated hPD-1 and mPD-1 (both produced in E. coli) are 50 pM and 25 pM, respectively (FIG. 5).

Next the binding property of IMT200 was compared to that of known PD-1 blockers on PD-1 (FIG. 6). Glycosylated human PD-1 His protein produced in mammalian cells (Abcam) (FIG. 6A) or non-glycosylated human PD-1 produced in E. coli (FIG. 6B) at 0.1 ug/ml was coated in 96-well plate overnight. After three times wash, plates were blocked with PBS buffer containing 2% BSA at room temperature for 1 hour. IMT200, Nivolumab (BioVision), Pembroilizumab (BioVision), at 3× serial dilution starting from 10 ug/m was added to the plates and incubated for one hour. Plates were then washed for three times and followed by incubation with anti-mouse or human IgG-HRP (Jackson Immuno Research) for 1 h at room temperature. The color was developed as stated above. The results show that IMT200 binds to both glycosylated and non-glycosylated hPD-1, while two known anti PD-1 antibodies, Nivolumab and Pembrolizumab, bind to glycosylated PD-1 only.

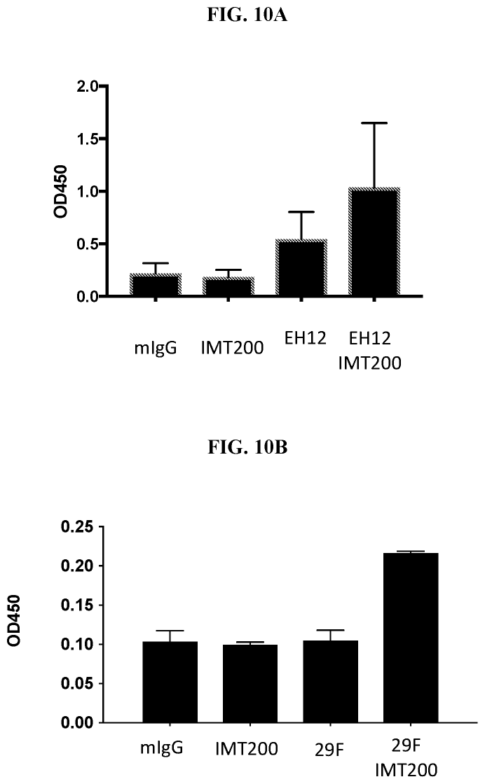

Finally, the IMT200 binding epitope on hPD-1 was mapped through screening a hPD-1 peptide array (FIG. 7 and Table 1). A peptide array containing 29 10 amino acid peptides with 5 amino acid overlapping derived from the human PD-1 protein sequence was synthesized (Genscript) and 20 ug of each peptide was coated in 96-well plate for overnight. After three times wash, plates were blocked with PBS buffer containing 2% BSA at room temperature for 1 hour. IMT200 1 ug/ml was added to the plates and incubated for one hour. Plates were then washed for three times and followed by incubation with anti-mouse IgG-HRP (Jackson Immuno Research) for 1 h at room temperature. The color was developed as stated above. Results indicated that peptide No 12 with sequence TDKLAAFPED (SEQ ID NO: 9) is the epitope through which IMT200 binds to hPD-1. The residue positions shown in Table 1 are in accordance to the position of the residues illustrated in SEQ ID NO: 4.

TABLE 1

| Peptide No. | | SEQ | |
|---|---|---|---|
| shown in | | ID | Residue |
| FIG. 7 | SEQUENCE | NO: | Positions |
| 1 | PGWFLDSPDR | 24 | 21-30 |
| 2 | DSPDRPWNPP | 25 | 26-35 |
| 3 | PWNPPTFSPA | 26 | 31-40 |
| 4 | TFSPALLVVT | 27 | 36-45 |
| 5 | LLVVTEGDNA | 28 | 41-50 |
| 6 | EGDNATFTCS | 29 | 46-55 |
| 7 | TFTCSFSNTS | 30 | 51-60 |
| 8 | FSNTSESFVL | 31 | 56-65 |
| 9 | ESFVLNWYRM | 32 | 61-70 |
| 10 | NWYRMSPSNQ | 33 | 66-75 |
| 11 | SPSNQTDKLA | 34 | 71-80 |
| 12 | TDKLAAFPED | 9 | 76-85 |
| 13 | AFPEDRSQPG | 35 | 81-90 |
| 14 | RSQPGQDCRF | 36 | 86-95 |
| 15 | QDCRFRVTQL | 37 | 91-100 |
| 16 | RVTQLPNGRD | 38 | 96-105 |
| 17 | PNGRDFHMSV | 39 | 101-110 |
| 18 | FHMSVVRARR | 40 | 106-115 |
| 19 | VRARRNDSGT | 41 | 111-120 |
| 20 | NDSGTYLCGA | 42 | 116-125 |
| 21 | YLCGAISLAP | 43 | 121-130 |
| 22 | ISLAPKAQIK | 44 | 126-135 |
| 23 | KAQIKESLRA | 45 | 131-140 |
| 24 | ESLRAELRVT | 46 | 136-145 |
| 25 | ELRVTERRAE | 47 | 141-150 |
| 26 | ERRAEVPTAH | 48 | 146-155 |
| 27 | VPTAHPSPSP | 49 | 151-160 |
| 28 | PSPSPRPAGQ | 50 | 156-165 |
| 29 | RPAGQFQTLV | 51 | 161-170 |

The above data confirms that monoclonal antibody IMT200 is a non-glycosylated PD-1 binder.

### Example 3 Blocking Assays—ELISA

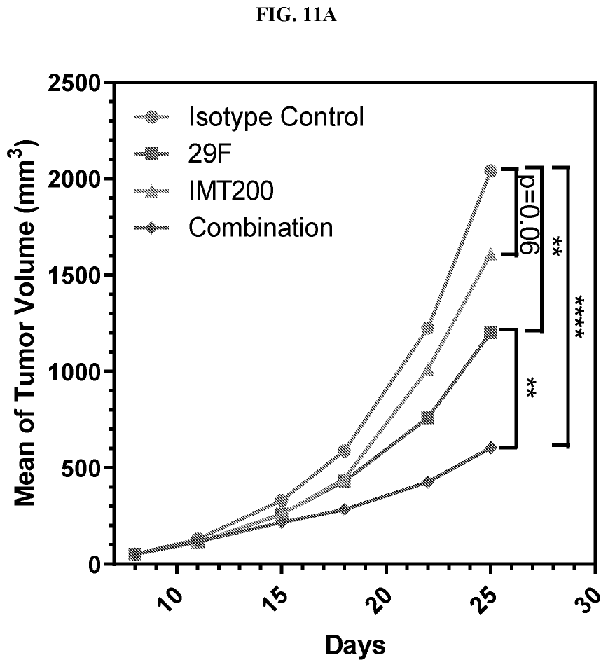

ELISA plates (ThermoFisher Scientific) were coated with hPD-1 His produced from mammalian cells (FIG. 8B) or non-glycosylated hPD-1 His protein produced from E. coli (FIG. 8A) in PBS and incubated at 4° C. for overnight. The plate was washed three times with TBST and then blocked with PBS buffer containing 2% BSA at room temperature for 1 hour. In FIG. 8A, IMT200 was added to wells that had been coated with non-glycosylated PD-1. The antibody was incubated for 10 minutes and deglycosylated mPD-1 Fc (R&D Systems) were then added to the plates and incubated for an additional one-hour. In FIG. 8B, IMT200 and anti hPD-1 mAb EH12 were separately added to well that has been coated with PD-1. The antibody was incubated for 10 minutes and hPD-L1 Fc (R&D Systems) was then added to the plates and incubated for an additional one-hour. Plates were then washed for three times and followed by incubation with anti-human-IgG-HRP (Jackson Immuno Research) for 1 h at room temperature. The color was developed as stated above. The results in FIG. 8A showed that IMT200 against hPD-1 blocked the interaction between PD-1 and PD-1, while it failed to block the binding between PD-1 and PD-L1, in contrast to the action of EH12 which blocked PD-1 binding to PD-L1, indicating PD-1 and PD-L1 bind PD-1 on different epitopes (FIG. 8B).

### Example 4. PD-1 Expression on Macrophages

Flow cytometry was used to detect PD-1 expression on macrophages. Mouse RAW macrophages (FIG. 9A), or human M1 macrophages (FIG. 9B), or human M2 macrophages (FIG. 9C), were incubated with biotin labeled IMT200 for 20 min on ice, followed by incubation with avidin PE antibody (Biolegend) for 20 min on ice. After wash, stained cells were analyzed using MACSquant Analyzer 10 (Miltenyi Biosci). The results in FIG. 9 showed that PD-1 expression was detected on RAW macrophages (FIG. 9A), human M2 macrophages (FIG. 9C), and to a less extent on human M1 macrophages (FIG. 9B).

### Example 5. PD-1 Function

Mixed Lymphocyte Reaction (MLR)

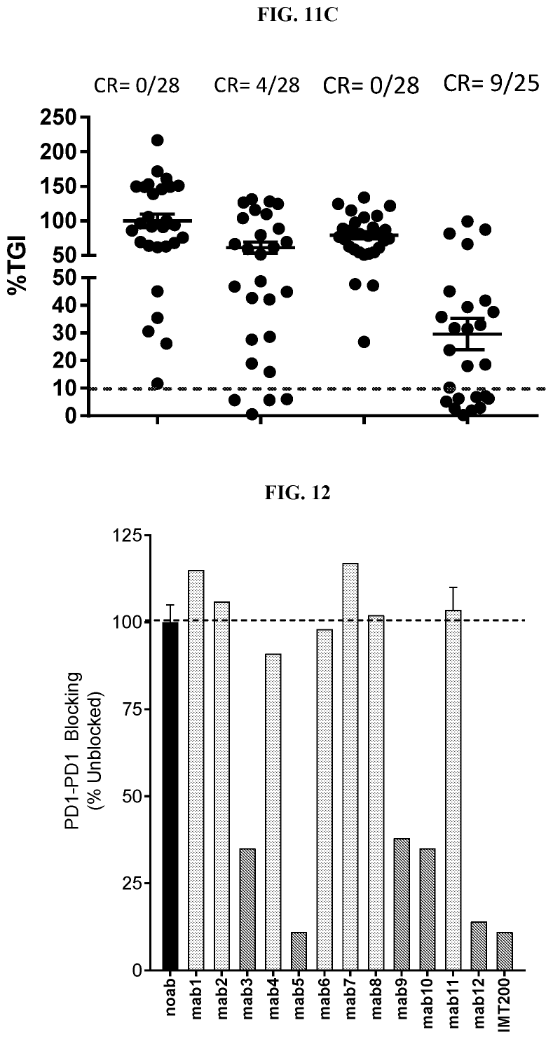

In FIG. 10A, human M1 macrophages from one donor were mixed with human CD4 T cells from another donor and were treated with 10 ug/ml control IgG, EH12 (BD bioscience), which binds to glycosylated PD-1 and blocks interaction between glycosylated PD-1 and PD-L1, or IMT200, or their combination for 8 days. Secreted IFNgamma was detected with an ELISA kit from eBioscience. The results in FIG. 10A showed that non-glycosylated PD-1 inhibitor mAb, IMT200, in combination with glycosylated PD1 inhibitor antibody, EH12, greatly enhanced the secretion of IFNgamma.

FIG. 10 shows the effect of IMT200 antibody in combination with existing PD-1 blocking antibodies on T cell activation. These two antibodies also bind glycosylated PD1 only. In FIG. 10A, 100,000 cells RAW macrophages were mixed with 100,000 mouse DO11.10 T cells in 100 μl medium. The mixture was placed to each well of flat 96-well plates. 50 ul medium containing IMT200 or mPD-1 antibody 29F at 80 ug/ml was added to each well. Then 50 μl medium containing OVA323-339 peptide (Invivogen) were added to the plates to a final concentration of 500 ng/ml per well. After overnight incubation, 100 μl supernatant was used for measuring IL-2 production of the T cells by ELISA (eBioscience). Results show that IMT200 in combination with 29F reversed PD-1 suppression by enhancing IL-2 production. Columns from left to right represent mIgG control, IMT200, 29F and combination of IMT200 and 29F, respectively.

### Example 6. An Anti-PD-1 Antibody Shows Anti-Tumor Activity in Mouse Primary Tumor Model

The anti-tumor efficacy of PD-1:PD-1 inhibitor in vivo was evaluated. The animal experiments were conducted according to a protocol approved by the Molecular Medicine Research Institute Institutional Animal Care and Use Committee. 7-week old female Balb/c mice were placed in a facility accredited by the Association for Assessment and Accreditation of Laboratory Animal Care upon arrival. On the day of tumor implantation, mice were anesthetized by inhalation anesthetic (3 to 5% Isoflurane in medical grade air). CT26.WT cells were washed and resuspended in PBS before subcutaneously injecting 2×10⁵ cells in 0.1 mL PBS using a syringe with a 25-ga needle. On day 7, the tumor volumes were measured and mice were randomly assigned into four groups (n=10). Mice were i.p. administered with 10 mg/Kg of mouse IgG2b (BioXCell) on day 7, 10, 14, 17 and 21, mPD1 antibody 29F (BioXCell) on day 7, 10 and 14, IMT200 antibody on day 7, 10, 14, 17 and 21, as well as combination of mPD1 antibody 29F (on day 7, 10 and 14) and IMT200 antibody (on day 7, 10, 14, 17 and 21). The animals were humanely sacrificed when tumor volume reached 3000 mm³. Results were expressed as mean tumor values. The statistical analysis was performed in comparison with IgG control group using one-way ANOVA.

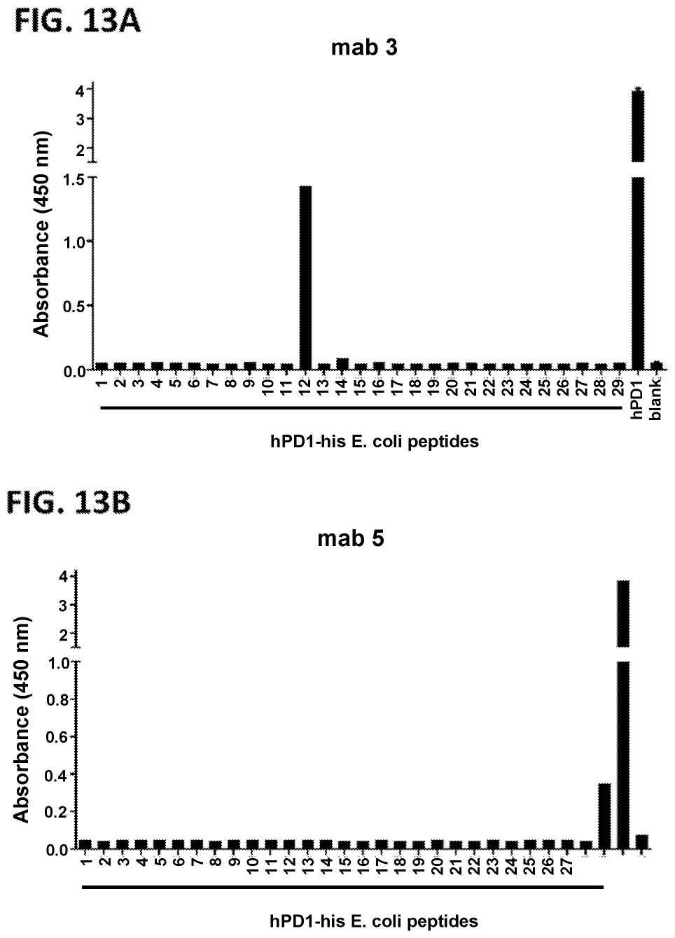

As shown in FIG. 11A, as compared to isotype or IMT200 or 29F single agent treated groups, the combination of IMT200 and 29F treated group showed significant reduction of tumor size. When plotted as individual tumor volumes, it was observed that a subset of 29F-treated animals exhibited more robust responses, with an increasing trend towards curative responses in animals treated with a combination of 29F and IMT200 (FIG. 11B). Tumor growth inhibition was calculated as a function of percent of mean vehicle-treated tumor volume, and animals with >90% tumor reductions were considered to be complete responses (CRs). Under these criteria, no animals treated with vehicle control or IMT200 exhibited CRs, whereas 4/28 animals treated with 29F single agent scored as CRs, and 9/25 animals treated with the combination of 29F and IMT200 exhibited CRs (FIG. 11C). Collectively, these data support the conclusion that deglycoylated-PD1 blocking antibody IMT200 can augment the activity of traditional PD1-PDL1 blockade.

### Example 7. Identification of Deglyco-PD1-Binding Antibodies with and without Deglyco-PD1-Deglyco-PD1 Blocking Activity

To identify deglycosylated PD1-targeted antibodies with the ability to block the interaction of PD1-degly and PD1, purified deglycosylated hPD1 and enzymatically deglycosylated mPD1-Fc proteins were incubated in the presence of various deglyacosylated PD1-targeted or control antibodies, or without antibody, and protein interaction was evaluated by ELISA. Deglycosylated Fc-tagged PD-1 was produced by incubating 10 ug of mPD1-Fc (R&D Systems, 1021-PD) in 10 ul with 2 ul 10× Protein Deglycosylation Mix II (New England Biolabs, P6044), 2 ul 10× Deglycosylation Mix Buffer 1 (New England Biolabs, B6044) and 6 ul H₂O and incubated for 30 minutes at room temperature, then overnight at 37 C, after which the protein was biotinylaed using the Thermo Scientific™ EZ-Link™ Sulfo-NHS-LC-Biotin No-Weigh™ Format kit (A39257). Purified human PD1 extracellular domain produced in E. coli fused to Fc (Ray Biotech) was diluted in phosphate buffered saline (PBS) (Corning) to a concentration of 2 μg/ml and 100 ul was added to each well of a 96-well ELISA plate (Thermo Fisher, 44-2404-21). After incubating the plate at 4° C. overnight, the plate was washed three times with 300 μl of PBS with 0.05% TWEEN (VWR) (PBST) per well. The plate was then blocked for an hour with 200 μl of 2% bovine serum albumin (BSA) (Sigma) in PBST per well at room temperature with gentle rocking. Thereafter, the 2% BSA in PBST was removed and 50 ul of antibody at 20 ug/ml in 2% BSA in PBST was added to the wells. The plate was incubated for 10 minutes at room temperature with gentle rocking. Afterwards, 50 ul of 2 ug/ml of biotinylated and enzymatically decglycosylated mouse PD-1 protein in 2% BSA in PBST was added to the wells. The plate was incubated for an hour at room temperature with gentle rocking. Thereafter, the plate was washed three times with 300 μl of PBST per well, and 100 ul of 0.3 ug/ml of Avidin-HRP (Jackson ImmunoResearch) in 2% BSA in PBST was added to each well. The plate was incubated for an hour with gentle rocking and then washed three times with 300 μl of PBST per well. 100 ul of avidin-HRP (1:1000) (Jackson ImmunoResearch) was then added to each well and the plate was incubated at room temperature for 30 minutes with gentle rocking. Thereafter, the plate was washed three times with 300 μl of PBST per well. 100 ul of TMB substrate (Fisher Scientific, 34029) was then added to each well. The reaction was stopped with 50 ul of 1 M HCl (VWR) per well. The plate was read using a plate reader (Molecular Devices) at absorbance of 450 nm. Percent blockade of deglyco-PD1-deglyco-PD1 interaction was calculated as the fraction of signal obtained in each experimental samples of the no antibody sample less background signal.

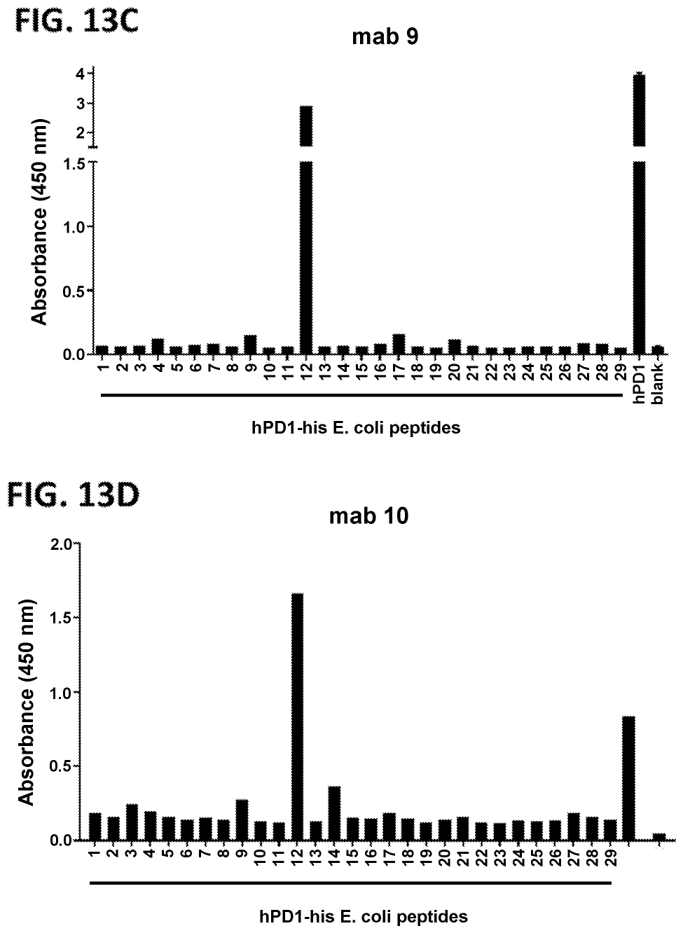

As shown in FIG. 12, deglyco-PD1-binding antibodies exhibited differential ability to block the interaction of deglyco-PD1 and deglyco-PD1. Deglyco-PD1-targeting mab4, mab6, mab7, mab8, and mab10 inhibited strongly, reducing binding to 17%, 27%, 29%, 27%, and 19% of unblocked controls, respectively. mab3, mab5, mab9, mab10, and mab12 and humanized mab IMT200 exhibited ability to interrupt deglyco-PD1-deglyco-PD1 binding, reducing the interaction to 65%, 89%, 62%, 65%, 86% and 89% of unblocked controls, respectively.

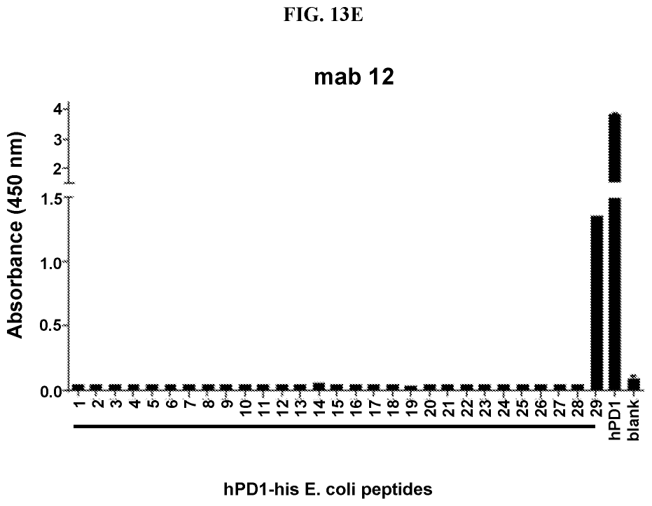

Table 2 illustrates the antibody names as shown in FIGS. 12-13E, the respective clone name, and the source of the antibody. Note that the antibodies generated from Immutics under the Vendor name are Applicant's antibodies generated de novo.

| ANTIBODY | VENDOR | CLONE |
|---|---|---|
| mab1 | Immutics | 2C1 |
| mab2 | Immutics | 3A4 |
| mab3 | Immutics | 3E5 |
| | Immutics | 3F12 |
| mab4 | Immutics | 3H11 |
| mab5 | BioLegend | 4F12 |
| mab6 | Immutics | 5B2 |
| mab7 | Immutics | 5B8 |
| mab8 | Immutics | 5E4 |
| mab9 | Immutics | 5E9 |
| mab10 | Immutics | 5G10 |
| mab11 | Novus | EH12.2 |
| mab12 | LS Bio | LS-C131333 |
| IMT200 | Immutics | IMT200 |

### Example 8. Deglyco-PD1-Targeted Antibodies with Deglyco-PD1-Deglyco-PD1 Blocking Activity Bind to Distinct Epitopes of PD1

To identify the epitopes to which deglyco-PD1 antibodies with and without deglyco-PD1-deglyco-PD1 blocking activity bound, a library of 20 amino acid peptides representing portions of the extracellular domain of PD1 was produced, and the ability to bind deglyco-PD1 antibodies was evaluated by ELISA. At least 2 ug/ml of deglyco-PD1 peptide in 50 ul of PBS or 0.1 ug/ml of full-length human deglyco-PD1 protein (Ray Biotech) in 100 ul of PBS was added to the wells of a 96-well ELISA plate (Thermo Fisher, 44-2404-21). After incubating the plate at 4° C. overnight, the plate was washed three times with 300 μl of PBST per well. The plate was then blocked for an hour with 200 μl of 2% BSA in PBST per well at room temperature with gentle rocking. Thereafter, the 2% BSA in PBST was removed and 100 ul of 0.1 ug/ml of antibody in 2% BSA in PBST was added to the wells. The plate was incubated for an hour at room temperature with gentle rocking and then washed three times with 300 μl of PBST per well. Afterwards, 100 ul of anti-mouse IgG-HRP (1:4000) (Jackson ImmunoResearch) or anti-rat IgG HRP (1:4000) (Jackson ImmunoResearch) was added to the wells. The plate was incubated for 30 minutes at room temperature with gentle rocking and then washed three times with 300 μl of PBST per well. 100 ul of TMB substrate (Fisher Scientific, 34029) was then added to each well. The reaction was stopped with 50 ul of 1 M HCl (VWR) per well. The plate was read using a plate reader (Molecular Devices) at absorbance of 450 nm.

As shown in FIG. 13A-FIG. 13E, deglyco-PD1-targeted antibodies mab3, mab9, mab10, and humanized mab IMT200 with deglyco-PD1-deglyco-PD1 blocking activity bound strongly to PD1 peptide 12, corresponding to the amino acid sequence TDKLAAFPED (SEQ ID NO: 9), comprising amino acid residues 76-85 of PD1 (SEQ ID NO: 4). Deglyco-PD1-deglyco-PD1 blocking activity, mab5 and mab12 bound to peptide 29, corresponding the amino acid sequence of RPAGQFQTLV (SEQ ID NO: 51), comprising amino acid residues 161-170 of PD1 (SEQ ID NO: 4). In contrast, deglcyo-PD1 bonding antibody without deglyco-PD1-deglyco-PD1 blocking activity mab11, failed to bind to any individual peptide. Thus, the epitopes comprised of amino acids TDKLAAFPED (SEQ ID NO: 9) or amino acids RPAGQFQTLV (SEQ ID NO: 51) define a previously unrecognized feature of PD1 that can provide utility in prospectively identifying antibodies with PD1-PD1 blocking activity.

### Example 9. Sequences

```
Mus musculus PD-1 NUCLEIC ACID (cDNA) SEQUENCE
Start and stop codons in Upper case.
SEQ ID NO: 1:
      1 tgagcagcgg ggaggaggaa gaggagactg ctactgaagg
cgacactgcc aggggctctg
    61 ggcATGtggg tccggcaggt accctggtca ttcacttggg
ctgtgctgca gttgagctgg
  121 caatcagggt ggcttctaga ggtccccaat gggccctgga
ggtccctcac cttctaccca
  181 gcctggctca cagtgtcaga gggagcaaat gccaccttca
cctgcagctt gtccaactgg
  241 tcggaggatc ttatgctgaa ctggaaccgc ctgagtccca
gcaaccagac tgaaaaacag
  301 gccgccttct gtaatggttt gagccaaccc gtccaggatg
cccgcttcca gatcatacag
  361 ctgcccaaca ggcatgactt ccacatgaac atccttgaca
cacggcgcaa tgacagtggc
  421 atctacctct gtggggccat ctccctgcac cccaaggcaa
aaatcgagga gagccctgga
  481 gcagagctcg tggtaacaga gagaatcctg gagacctcaa
caagatatcc cagcccctcg
  541 cccaaaccag aaggccggtt tcaaggcatg gtcattggta
tcatgagtgc cctagtgggt
  601 atccctgtat tgctgctgct ggcctgggcc ctagctgtct
tctgctcaac aagtatgtca
  661 gaggccagag gagctggaag caaggacgac actctgaagg
aggagccttc agcagcacct
  721 gtccctagtg tggcctatga ggagctggac ttccagggac
gagagaagac accagagctc
  781 cctaccgcct gtgtgcacac agaatatgcc accattgtct
tcactgaagg gctgggtgcc
  841 tcggccatgg gacgtagggg ctcagctgat ggcctgcagg
gtcctcggcc tccaagacat
  901 gaggatggac attgttcttg gcctcttTGA ccagattctt
cagccattag catgctgcag
  961 accctccaca gagagcaccg gtccgtccct cagtcaagag
gagcatgcag gctacagttc
1021 agccaaggct cccagggtct gagctagctg gagtgacagc
ccagcgcctg caccaattcc
1081 agcacatgca ctgttgagtg agagctcact tcaggtttac
cacaagctgg gagcagcagg
1141 cttcccggtt tcctattgtc acaaggtgca gagctggggc
ctaagcctat gtctcctgaa
1201 tcctactgtt gggcacttct agggacttga gacactatag
ccaatggcct ctgtgggttc
1261 tgtgcctgga aatggagaga tctgagtaca gcctgctttg
aatggccctg tgaggcaacc
1321 ccaaagcaag ggggtccagg tatactatgg gcccagcacc
taaagccacc cttgggagat
1381 gatactcagg tgggaaattc gtagactggg ggactgaacc
aatcccaaga tctggaaaag
1441 ttttgatgaa gacttgaaaa gctcctagct tcgggggtct
gggaagcatg agcacttacc
1501 aggcaaaagc tccgtgagcg tatctgctgt ccttctgcat
gcccaggtac ctcagttttt
1561 ttcaacagca aggaaactag ggcaataaag ggaaccagca
gagctagagc cacccacaca
1621 tccagggggg cacttgactc tccctactcc tcctaggaac
caaaaggaca aagtccatgt
1681 tgacagcagg gaaggaaagg gggatataac cttgacgcaa
accaacactg gggtgttaga
1741 atctcctcat tcactctgtc ctggagttgg gttctggctc
tccttcacac ctaggactct
1801 gaaatgagca agcacttcag acagtcaggg tagcaagagt
ctagctgtct ggtgggcacc
1861 caaaatgacc agggcttaag tccctttcct ttggtttaag
cccgttataa ttaaatggta
1921 ccaaaagctt taaaaaaaaa aaaaaaaaaa aaaaaaaaaa
aaaaaaaaaa aaa
Mus musculus PD-1 PROTEIN SEQUENCE
SEQ ID NO 2:
MWVRQVPWSFTWAVLQLSWQSGWLLEVPNGPWRSLTFYPAWLTVSEGANA
TFTCSLSNWSEDLMLNWNRLSPSNQTEKQAAFCNGLSQPVQDARFQIIQL
PNRHDEHMNILDTRRNDSGIYLCGAISLHPKAKIEESPGAELVVTERILE
TSTRYPSPSPKPEGREQGMVIGIMSALVGIPVLLLLAWALAVECSTSMSE
ARGAGSKDDTLKEEPSAAPVPSVAYEELDFQGREKTPELPTACVHTEYAT
IVFTEGLGASAMGRRGSADGLQGPRPPRHEDGHCSWPL
Homo sapiens PD-1 NUCLEIC ACID (cDNA) SEQUENCE
start and stop codons in Upper case
SEQ ID NO: 3:
      1 agtttccctt ccgctcacct ccgcctgagc agtggagaag
gcggcactct ggtggggctg
    61 ctccaggcAT Gcagatccca caggcgccct ggccagtcgt
ctgggcggtg ctacaactgg
  121 gctggcggcc aggatggttc ttagactccc cagacaggcc
ctggaacccc cccaccttct
  181 ccccagccct gctcgtggtg accgaagggg acaacgccac
cttcacctgc agcttctcca
  241 acacatcgga gagcttcgtg ctaaactggt accgcatgag
ccccagcaac cagacggaca
  301 agctggccgc cttccccgag gaccgcagcc agcccggcca
ggactgccgc ttccgtgtca
  361 cacaactgcc caacgggcgt gacttccaca tgagcgtggt
cagggcccgg cgcaatgaca
  421 gcggcaccta cctctgtggg gccatctccc tggcccccaa
ggcgcagatc aaagagagcc
  481 tgcgggcaga gctcagggtg acagagagaa gggcagaagt
gcccacagcc caccccagcc
  541 cctcacccag gccagccggc cagttccaaa ccctggtggt
tggtgtcgtg ggcggcctgc
  601 tgggcagcct ggtgctgcta gtctgggtcc tggccgtcat
ctgctcccgg gccgcacgag
  661 ggacaatagg agccaggcgc accggccagc ccctgaagga
ggacccctca gccgtgcctg
  721 tgttctctgt ggactatggg gagctggatt tccagtggcg
agagaagacc ccggagcccc
  781 ccgtgccctg tgtccctgag cagacggagt atgccaccat
tgtctttcct agcggaatgg
  841 gcacctcatc ccccgcccgc aggggctcag ctgacggccc
tcggagtgcc cagccactga
  901 ggcctgagga tggacactgc tcttggcccc tcTGAccggc
ttccttggcc accagtgttc
  961 tgcagaccct ccaccatgag cccgggtcag cgcatttcct
caggagaagc aggcagggtg
1021 caggccattg caggccgtcc aggggctgag ctgcctgggg
gcgaccgggg ctccagcctg
1081 cacctgcacc aggcacagcc ccaccacagg actcatgtct
caatgcccac agtgagccca
1141 ggcagcaggt gtcaccgtcc cctacaggga gggccagatg
cagtcactgc ttcaggtcct
1201 gccagcacag agctgcctgc gtccagctcc ctgaatctct
gctgctgctg ctgctgctgc
1261 tgctgctgcc tgcggcccgg ggctgaaggc gccgtggccc
tgcctgacgc cccggagcct
1321 cctgcctgaa cttgggggct ggttggagat ggccttggag
cagccaaggt gcccctggca
1381 gtggcatccc gaaacgccct ggacgcaggg cccaagactg
ggcacaggag tgggaggtac
1441 atggggctgg ggactcccca ggagttatct gctccctgca
ggcctagaga agtttcaggg
1501 aaggtcagaa gagctcctgg ctgtggtggg cagggcagga
aacccctcca cctttacaca
1561 tgcccaggca gcacctcagg ccctttgtgg ggcagggaag
ctgaggcagt aagcgggcag
1621 gcagagctgg aggcctttca ggcccagcca gcactctggc
ctcctgccgc cgcattccac
1681 cccagcccct cacaccactc gggagaggga catcctacgg
tcccaaggtc aggagggcag
1741 ggctggggtt gactcaggcc cctcccagct gtggccacct
gggtgttggg agggcagaag
1801 tgcaggcacc tagggccccc catgtgccca ccctgggagc
tctccttgga acccattcct
1861 gaaattattt aaaggggttg gccgggctcc caccagggcc
tgggtgggaa ggtacaggcg
1921 ttcccccggg gcctagtacc cccgccgtgg cctatccact
cctcacatcc acacactgca
1981 cccccactcc tggggcaggg ccaccagcat ccaggcggcc
agcaggcacc tgagtggctg
2041 ggacaaggga tcccccttcc ctgtggttct attatattat
aattataatt aaatatgaga
2101 gcatgctaag gaaaa
Homo sapiens PD-1 PROTEIN SEQUENCE
SEQ ID NO 4:
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL
SEQ ID NO 5: IMT200
Heavy chain: DNA sequence (408 bp)
Leader sequence-FR1-CDR1-FR2-CDR2-FR3-CDR3-FR4
ATGGAATGGCCTTGTATCTTTCTCTTCCTCCTGTCAGTAACTGAAGGTGT
CCACTCCCAGGTTCAGCTGCAGCAGTCTGGACCTGAGCTGGTGAAGCCTG
GGGCCTCAGTGAAGATTTCCTGCAAGGCTTCTGGCTTCGCATTCAGTGGC
TCCTGGATGAACTGGATGAAGCAGAGGCCTGGAAAGGGTCTTGAGTGGAT
TGGACGGATTTATCCTGGAGATGGAGATACTAACTACAATGGGAAGTCCA
AGGGCAAGGCCACACTTACTGCAGACACATCCTCCAGCACAGCCTACATG
CAACTCAGCAGCCTGACATCTGAGGACTCTGCGGTCTACTTCTGTACAAG
ATCAACTACGATATTAGCAGACTACTGGGGCCAAGGCACCACTCTCACAG
TCTCCTCA
SEQ ID NO 6: IMT200
Heavy chain: Amino acids sequence (136 aa)
Leader sequence-FR1-CDR1-FR2-CDR2-FR3-CDR3-FR4
MEWPCIFLFLLSVTEGVHSQVQLQQSGPELVKPGASVKISCKASGFAFSG
SWMNWMKQRPGKGLEWIGRIYPGDGDTNYNGKSKGKATLTADTSSSTAYM
QLSSLTSEDSAVYFCTRSTTILADYWGQGTTLTVSS
SEQ ID NO 7: IMT200
Light chain: DNA sequence (381 bp)
Leader sequence-FR1-CDR1-FR2-CDR2-FR3-CDR3-FR4
ATGATGTCCTCTGCTCAGTTCCTTGGTCTCCTGTTGCTCTGTTTGCAAGG
TACCAGATGTGATATCCAGATGACACAGACTACATCCTCCCTGTCTGCCT
CTCTGGGAGACAGAGTCACCATCAGTTGCAGGGCAAGTCAGGACATTGCC
AATTATTTAAACTGGTATCAGCAGAAACCAGATGGAACTGTTAAACTCCT
GATCTACTACACATCAAGAAAATATTCAGGAGTCCCATCAAGGTTCAGTG
GCAGTGGGTCTGGAACAGATTATTCTCTCACCATTAGCAACCTGGACCAA
GAAGATATTGCCACTTACTTTTGCCAACAGGGTAAAACGCTTCCGTGGAC
GTTCGGTGGAGGCACCAAGCTGGAAATCAAA
SEQ ID NO 8: IMT200
Light chain: Amino acids sequence (127 aa)
Leader sequence-FR1-CDR1-FR2-CDR2-FR3-CDR3-FR4
MMSSAQFLGLLLLCLQGTRCDIQMTQTTSSLSASLGDRVTISCRASQDIA
NYLNWYQQKPDGTVKLLIYYTSRKYSGVPSRFSGSGSGTDYSLTISNLDQ
EDIATYFCQQGKTLPWTFGGGTKLEIK
```

Table 3 illustrates the sequences in the heavy chain CDR and frame work regions.

| SEQ | | |
|---|---|---|
| ID | | |
| NO: | IMT200 | Sequence |
| 10 | Heavy chain FR1 | QVQLQQSGPELVKPGAS |
| | | VKISCKASGFAFS |
| 11 | Heavy chain CDR1 | GSWMN |
| 12 | Heavy chain FR2 | WMKQRPGKGLEWIG |
| 13 | Heavy chain CDR2 | RIYPGDGDTNYNGKSKG |
| 14 | Heavy chain FR3 | KATLTADTSSSTAYMQL |
| | | SSLTSEDSAVYFCTR |
| 15 | Heavy chain CDR3 | STTILADY |
| 16 | Heavy chain FR4 | WGQGTTLTVSS |

Table 4 illustrates the sequences in the light chain CDR and frame work regions.

| SEQ | | |
|---|---|---|
| ID | | |
| NO: | IMT200 | Sequence |
| 17 | Light chain FR1 | DIQMTQTTSSLSASL |
| | | GDRVTISC |
| 18 | Light chain CDR1 | RASQDIANYLN |
| 19 | Light chain FR2 | WYQQKPDGTVKLLIY |
| 20 | Light chain CDR2 | YTSRKYS |
| 21 | Light chain FR3 | GVPSRFSGSGSGTDY |
| | | SLTISNLDQEDIATYFC |
| 22 | Light chain CDR3 | QQGKTLPWT |
| 23 | Light chain FR4 | FGGGTKLEIK |

Embodiment 1 describes an antibody that impairs an interaction between two non-glycosylated PD-1 polypeptides, wherein the antibody comprises three variable heavy chain complementarity-determining regions (CDRs) and three variable light chain CDRs, and wherein the antibody specifically binds to a region of the non-glycosylated PD-1 polypeptide comprising SEQ ID NO: 9.

### Embodiment 2

The antibody of embodiment 1, wherein the antibody does not impair an interaction between a PD-1 polypeptide and a programmed cell death ligand.

### Embodiment 3

The antibody of embodiment 1 or 2, wherein the antibody further binds to a glycosylated PD-1 polypeptide.

### Embodiment 4

The antibody of embodiment 3, wherein a binding affinity of the antibody to the glycosylated PD-1 polypeptide is equivalent to a binding affinity of a control to the glycosylated PD-1 polypeptide.

### Embodiment 5

The antibody of embodiment 4, wherein the control is Nivolumab or Pembrolizumab.

### Embodiment 6

The antibody of any one of the embodiments 1-5, wherein the antibody impairs the interaction of the two non-glycosylated PD-1 polypeptides by at least 50%, 60%, 70%, 80%, 90%, or more.

### Embodiment 7

The antibody of any one of the embodiments 1-6, wherein the antibody binds to a human non-glycosylated PD-1 polypeptide, a mouse non-glycosylated PD-1 polypeptide, or a combination thereof.

### Embodiment 8

The antibody of any one of the embodiments 1-7, wherein the antibody comprises three variable heavy chain complementarity-determining regions (CDRs) and three variable light chain CDRs, and wherein the three heavy chain CDRs comprise SEQ ID NOs: 11, 13, and 15, respectively.

### Embodiment 9

The antibody of embodiment 8, wherein the three light chain CDRs comprise SEQ ID NOs: 18, 20, and 22, respectively.

### Embodiment 10

The antibody of any one of the embodiments 1-9, wherein the antibody comprises a heavy chain variable region (VH) comprising SEQ ID NO: 7.

### Embodiment 11

The antibody of any one of the embodiments 1-10, wherein the antibody or its binding fragments thereof comprises a light chain variable region (VL) comprising SEQ ID NO: 8.

### Embodiment 12

The antibody of any one of the embodiments 1-7, wherein the antibody comprises three heavy chain CDRs selected from the heavy chain CDRs of mab3, mab9, and mab10, and three light chain CDRs selected from the light chain CDRs of mab3, mab9, and mab10.

### Embodiment 13

The antibody of any one of the embodiments 1-12, wherein the antibody is a humanized antibody or binding fragments thereof.

### Embodiment 14

The antibody of any one of the embodiments 1-13, wherein the antibody comprises a monoclonal antibody or binding fragments thereof.

### Embodiment 15

The antibody of any one of the embodiments 1-14, wherein the antibody comprises a monovalent Fab′, a divalent Fab2, a single-chain variable fragment (scFv), a diabody, a minibody, a nanobody, a single-domain antibody (sdAb), or a camelid antibody or binding fragment thereof

### Embodiment 16

The antibody of any one of the embodiments 1-15, wherein the antibody comprises a bispecific antibody or its binding fragments thereof.

### Embodiment 17

The antibody of any one of the embodiments 1-16, wherein the antibody is a full-length antibody, optionally comprising an Fc region selected from IgG1, IgG2, or IgG4.

### Embodiment 18

The antibody of any one of the embodiments 1-7 or 12, wherein the antibody is IMT200.

### Embodiment 19

The antibody of any one of the embodiments 1-7 or 12, wherein the antibody is mab3.

### Embodiment 20

The antibody of any one of the embodiments 1-7 or 12, wherein the antibody is mab9.

### Embodiment 21

The antibody of any one of the embodiments 1-7 or 12, wherein the antibody is mab10.

### Embodiment 22

The antibody of embodiment 2, wherein the programmed cell death ligand is PD-1 ligand 1 (PD-L1) or PD-1 ligand 2 (PD-L2).

### Embodiment 23

The antibody of any one of the embodiments 1-22, wherein the antibody is an isolated antibody.

Embodiment 24 describes a pharmaceutical combination comprising: a non-glycosylated PD-1 inhibitor that impairs an interaction between two non-glycosylated PD-1 polypeptides; a glycosylated PD-1 inhibitor that impairs an interaction between a glycosylated PD-1 polypeptide and a programmed cell death ligand; and a pharmaceutically acceptable vehicle or excipient.

### Embodiment 25

The pharmaceutical combination of embodiment 24, wherein the non-glycosylated PD-1 inhibitor binds to a region of the non-glycosylated PD-1 polypeptide comprising SEQ ID NO: 9.

### Embodiment 26

The pharmaceutical combination of embodiment 24 or 25, wherein the non-glycosylated PD-1 inhibitor does not impair an interaction between a PD-1 polypeptide and the programmed cell death ligand.

### Embodiment 27

The pharmaceutical combination of any one of the embodiments 24-26, wherein the non-glycosylated PD-1 inhibitor further binds to a glycosylated PD-1 polypeptide.

### Embodiment 28

The pharmaceutical combination of embodiment 27, wherein a binding affinity of the non-glycosylated PD-1 inhibitor to the glycosylated PD-1 polypeptide is equivalent to a binding affinity of a control to the glycosylated PD-1 polypeptide.

### Embodiment 29

The pharmaceutical combination of embodiment 28, wherein the control is Nivolumab or Pembrolizumab.

### Embodiment 30

The pharmaceutical combination of any one of the embodiments 24-29, wherein the non-glycosylated PD-1 inhibitor binds to human non-glycosylated PD-1 polypeptide, mouse non-glycosylated PD-1 polypeptide, or a combination thereof.

### Embodiment 31

The pharmaceutical combination of any one of the embodiments 24-30, wherein the non-glycosylated PD-1 inhibitor is an antibody, optionally an isolated antibody.

### Embodiment 32

The pharmaceutical combination of embodiment 31, wherein the antibody is a monoclonal antibody or its binding fragments thereof.

### Embodiment 33

The pharmaceutical combination of embodiment 31 or 32, wherein the antibody is a humanized antibody or its binding fragments thereof.

### Embodiment 34

The pharmaceutical combination of any one of the embodiments 31-33, wherein the antibody comprises a monovalent Fab′, a divalent Fab2, a single-chain variable fragment (scFv), a diabody, a minibody, a nanobody, a single-domain antibody (sdAb), or a camelid antibody or binding fragment thereof.

### Embodiment 35

The pharmaceutical combination of any one of the embodiments 31-34, wherein the antibody comprises a bispecific antibody or its binding fragments thereof.

### Embodiment 36

The pharmaceutical combination of any one of the embodiments 31-35, wherein the antibody comprises three variable heavy chain complementarity-determining regions (CDRs) comprising SEQ ID NOs: 11, 13, and 15; and three variable light chain CDRs comprising SEQ ID NOs: 18, 20, and 22.

### Embodiment 37

The pharmaceutical combination of any one of the embodiments 31-36, wherein the antibody comprises a heavy chain variable region (VH) comprising SEQ ID NO: 7.

### Embodiment 38

The pharmaceutical combination of any one of the embodiments 31-37, wherein the antibody comprises a light chain variable region (VL) comprising SEQ ID NO: 8.

### Embodiment 39

The pharmaceutical combination of any one of the embodiments 31-38, wherein the antibody is IMT200.

### Embodiment 40

The pharmaceutical combination of any one of the embodiments 31-35, wherein the antibody comprises three heavy chain CDRs selected from the heavy chain CDRs of mab3, mab9, and mab10, and three light chain CDRs selected from the light chain CDRs of mab3, mab9, and mab10.

### Embodiment 41

The pharmaceutical combination of any one of the embodiments 31-35 or 40, wherein the antibody is mab3, mab9, or mab10.

### Embodiment 42

The pharmaceutical combination of embodiment 24, wherein the programed cell death ligand is PD-1 ligand 1 (PD-L1).

### Embodiment 43

The pharmaceutical combination of embodiment 24, wherein the programed cell death ligand is PD-1 ligand 2 (PD-L2).

### Embodiment 44

The pharmaceutical combination of any one of the embodiments 24-43, wherein the glycosylated PD-1 inhibitor is an antibody or its binding fragments thereof.

### Embodiment 45

The pharmaceutical combination of embodiment 44, wherein the glycosylated PD-1 inhibitor is Nivolumab or Pembrolizumab.

### Embodiment 46

The pharmaceutical combination of embodiment 44, wherein the glycosylated PD-1 inhibitor is EH12 or 29F.

### Embodiment 47

The pharmaceutical combination of any one of the embodiments 24-46, wherein the pharmaceutical combination is formulated for systemic administration.

### Embodiment 48

The pharmaceutical combination of any one of the embodiments 24-46, wherein the pharmaceutical combination is formulated for local administration.

### Embodiment 49

The pharmaceutical combination of any one of the embodiments 24-48, wherein the pharmaceutical combination is formulated for parenteral administration.

### Embodiment 50

The pharmaceutical combination of any one of the embodiments 24-49, wherein the pharmaceutical combination is formulated as a pharmaceutical composition.

### Embodiment 51

The pharmaceutical combination of any one of the embodiments 24-49, wherein the pharmaceutical combination is formulated as separate dosages.

Embodiment 52 describes a method of disrupting an interaction between two non-glycosylated PD-1 polypeptides, comprising: contacting a non-glycosylated PD-1 inhibitor to a plurality of cells comprising two cells each expressing a non-glycosylated PD-1 polypeptide, wherein the non-glycosylated PD-1 inhibitor impairs the interaction between the two non-glycosylated PD-1 polypeptides.

### Embodiment 53

The method of embodiment 52, wherein the two cells are located within a tumor microenvironment (TME).

Embodiment 54 describes a method of activating an immune response in a subject in need thereof, comprising: administering to the subject a non-glycosylated PD-1 inhibitor and a glycosylated PD-1 inhibitor to activate the immune response, wherein the non-glycosylated PD-1 inhibitor impairs an interaction between two non-glycosylated PD-1 polypeptides, and wherein the glycosylated PD-1 inhibitor impairs an interaction between a glycosylated PD-1 polypeptide and a programmed cell death ligand.

### Embodiment 55

The method of embodiment 54, wherein each of the two non-glycosylated PD-1 polypeptides is expressed on a cell and the two cells are different.

### Embodiment 56

The method of embodiment 55, wherein the two cells are located within a tumor microenvironment (TME).

### Embodiment 57

The method of embodiment 54, wherein the programmed cell death ligand is PD-1 ligand 1 (PD-L1).

### Embodiment 58

The method of embodiment 54, wherein the programmed cell death ligand is PD-1 ligand 2 (PD-L2).

Embodiment 59 describes a method of reducing tumor cells within a tumor microenvironment (TME) in a subject, comprising: contacting a plurality of cells located within the TME with a non-glycosylated PD-1 inhibitor and a glycosylated PD-1 inhibitor.

### Embodiment 60

The method of embodiment 59, wherein the tumor cells are reduced by at least 5%, 10%, 15%, 20%, 25%, 30%, 35%, 40%, 45%, 50%, 60%, 70%, 80%, or 90%.

### Embodiment 61

The method of embodiment 59, wherein the subject is diagnosed with a cancer.

### Embodiment 62

The method of embodiment 61, wherein the cancer is a solid tumor.

### Embodiment 63

The method of embodiment 62, wherein the solid tumor is breast cancer, bile duct cancer, bladder cancer, colorectal cancer, gastric cancer, liver cancer, multiple myeloma, ovarian cancer, pancreatic cancer, prostate cancer, or thyroid cancer.

### Embodiment 64

The method of embodiment 61, wherein the cancer is a hematologic malignancy.

### Embodiment 65

The method of any one of the embodiments 61-64, wherein the cancer is a metastatic cancer.

### Embodiment 66

The method of any one of the embodiments 61-64, wherein the cancer is a relapsed or refractory cancer.

### Embodiment 67

The method of embodiment 59, wherein the non-glycosylated PD-1 inhibitor impairs an interaction between two non-glycosylated PD-1 polypeptides.

### Embodiment 68

The method of any one of the embodiments 52-67, wherein the interaction is impaired by about 20%, 30%, 40%, 50%, 60%, 70%, 80%, 90%, 95%, or 100%.

### Embodiment 69

The method of any one of the embodiments 52-67, wherein the interaction is impaired by about 1-fold, 2-fold, 3-fold, 4-fold, 5-fold, 10-fold, 20-fold, 30-fold, 40-fold, 50-fold, 100-fold, 200-fold, 500-fold, or more.

### Embodiment 70

The method of any one of the embodiments 52-67, wherein the non-glycosylated PD-1 inhibitor blocks the interaction between two non-glycosylated PD-1 polypeptides.

### Embodiment 71

The method of any one of the embodiments 52-70, wherein the non-glycosylated PD-1 inhibitor binds to a region of the non-glycosylated PD-1 polypeptide comprising SEQ ID NO: 9.

### Embodiment 72

The method of any one of the embodiments 52-71, wherein the non-glycosylated PD-1 inhibitor does not impair an interaction between a PD-1 polypeptide and the programmed cell death ligand.

### Embodiment 73

The method of any one of the embodiments 52-72, wherein the non-glycosylated PD-1 inhibitor further binds to a glycosylated PD-1 polypeptide.

### Embodiment 74

The method of embodiment 73, wherein a binding affinity of the non-glycosylated PD-1 inhibitor to the glycosylated PD-1 polypeptide is equivalent to a binding affinity of a control to the glycosylated PD-1 polypeptide.

### Embodiment 75

The method of embodiment 74, wherein the control is Nivolumab or Pembrolizumab.

### Embodiment 76

The method of any one of the embodiments 52-75, wherein the non-glycosylated PD-1 inhibitor binds to human non-glycosylated PD-1 polypeptide, mouse non-glycosylated PD-1 polypeptide, or a combination thereof.

### Embodiment 77

The method of any one of the embodiments 52-76, wherein the non-glycosylated PD-1 inhibitor is an antibody.

### Embodiment 78

The method of embodiment 77, wherein the antibody is a monoclonal antibody or its binding fragments thereof.

### Embodiment 79

The method of embodiment 77 or 78, wherein the antibody is a humanized antibody or its binding fragments thereof.

### Embodiment 80

The method of any one of the embodiments 77-79, wherein the antibody comprises a monovalent Fab′, a divalent Fab2, a single-chain variable fragment (scFv), a diabody, a minibody, a nanobody, a single-domain antibody (sdAb), or a camelid antibody or binding fragment thereof.

### Embodiment 81

The method of any one of the embodiments 77-80, wherein the antibody is a bispecific antibody or its binding fragments thereof.

### Embodiment 82

The method of any one of the embodiments 77-81, wherein the antibody comprises three variable heavy chain complementarity-determining regions (CDRs) comprising SEQ ID NOs: 11, 13, and 15; and three variable light chain CDRs comprising SEQ ID NOs: 18, 20, and 22.

### Embodiment 83

The method of any one of the embodiments 77-82, wherein the antibody comprises a heavy chain variable region (VH) comprising SEQ ID NO: 7.

### Embodiment 84

The method of any one of the embodiments 77-83, wherein the antibody comprises a light chain variable region (VL) comprising SEQ ID NO: 8.

### Embodiment 85

The method of any one of the embodiments 77-84, wherein the antibody is IMT200.

### Embodiment 86

The method of any one of the embodiments 77-81, wherein the antibody comprises three heavy chain CDRs selected from the heavy chain CDRs of mab3, mab9, and mab10, and three light chain CDRs selected from the light chain CDRs of mab3, mab9, and mab10.

### Embodiment 87

The method of any one of the embodiments 77-81 or 86, wherein the antibody is mab3, mab9, or mab10.

### Embodiment 88

The method of any one of the embodiments 52-87, wherein the glycosylated PD-1 inhibitor is an antibody or its binding fragments thereof.

### Embodiment 89

The method of embodiment 88, wherein the glycosylated PD-1 inhibitor is Nivolumab or Pembrolizumab.

### Embodiment 90

The method of embodiment 88, wherein the glycosylated PD-1 inhibitor is EH12 or 29F.

### Embodiment 91

The method of any one of the embodiments 54-90, wherein a combination of the non-glycosylated PD-1 inhibitor and the glycosylated PD-1 inhibitor enhances the production of a cytokine in the subject.

### Embodiment 92

The method of embodiment 91, wherein the enhanced production of the cytokine is compared to a production level of the cytokine by either the non-glycosylated PD-1 inhibitor alone or the glycosylated PD-1 inhibitor alone.

### Embodiment 93

The method of embodiment 91, wherein the cytokine is interleukin 2 (IL-2) or interferon gamma (IFNγ).

### Embodiment 94

The method of any one of the embodiments 52-93, wherein the antibody is formulated for systemic administration.

### Embodiment 95

The method of any one of the embodiments 52-94, wherein the antibody is formulated for local administration.

### Embodiment 96

The method of any one of the embodiments 52-95, wherein the antibody is formulated for parenteral administration.

Embodiment 97 describes a method of activating an immune response in a subject in need thereof, comprising: administering to the subject a non-glycosylated PD-1 inhibitor to activate the immune response, wherein the non-glycosylated PD-1 inhibitor impairs an interaction between two non-glycosylated PD-1 polypeptides.

Embodiment 98 describes a method of reducing tumor cells within a tumor microenvironment (TME) in a subject, comprising: contacting a plurality of cells located within the TME with a non-glycosylated PD-1 inhibitor.

### Embodiment 99

The method of any of the preceding embodiments, wherein the subject is a human.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### REFERENCE TO SEQUENCE LISTING

The present application is being filed along with a Sequence Listing in electronic format. The Sequence Listing is provided as a file entitled IMMUT011NP.TXT, which was created and last modified on Sep. 24, 2020, which is 25,627 bytes in size. The information in the electronic Sequence Listing is hereby incorporated by reference in its entirety.

1. An antibody that impairs an interaction between two non-glycosylated PD-1 polypeptides, wherein the antibody comprises three variable heavy chain complementarity-determining regions (CDRs) and three variable light chain CDRs, and wherein the antibody specifically binds to a region of the non-glycosylated PD-1 polypeptide comprising SEQ ID NO: 9.

2. The antibody of claim 1, wherein the antibody does not impair an interaction between a PD-1 polypeptide and a programmed cell death ligand.

3. The antibody of claim 1, wherein the antibody impairs the interaction of the two non-glycosylated PD-1 polypeptides by at least 50%, 60%, 70%, 80%, 90%, or more.

4. The antibody of claim 1, wherein the antibody binds to a human non-glycosylated PD-1 polypeptide, a mouse non-glycosylated PD-1 polypeptide, or a combination thereof.

5. The antibody of claim 1, wherein the antibody comprises three variable heavy chain complementarity-determining regions (CDRs) and three variable light chain CDRs, wherein the three heavy chain CDRs comprise SEQ ID NOs: 11, 13, and 15, respectively, and wherein the three light chain CDRs comprise SEQ ID NOs: 18, 20, and 22, respectively.

6. The antibody of claim 1, wherein the antibody comprises three heavy chain CDRs selected from the heavy chain CDRs of mab3, mab9, and mab10, and three light chain CDRs selected from the light chain CDRs of mab3, mab9, and mab10.

7. The antibody of claim 1, wherein the antibody is a humanized antibody or binding fragments thereof.

8. The antibody of claim 1, wherein the antibody comprises a monoclonal antibody or binding fragments thereof.

9. The antibody of claim 1, wherein the antibody comprises a monovalent Fab′, a divalent Fab2, a single-chain variable fragment (scFv), a diabody, a minibody, a nanobody, a single-domain antibody (sdAb), or a camelid antibody or binding fragment thereof.

10. The antibody of claim 1, wherein the antibody comprises a bispecific antibody or its binding fragments thereof.

11. The antibody of claim 1, wherein the antibody is a full-length antibody, optionally comprising an Fc region selected from IgG1, IgG2, or IgG4.

12. The antibody of claim 1, wherein the antibody is IMT200, mab3, mab9, or mab10.

13. The antibody of claim 2, wherein the programmed cell death ligand is PD-1 ligand 1 (PD-L1) or PD-1 ligand 2 (PD-L2).

14. A pharmaceutical combination comprising:
an antibody of claim 1;
a glycosylated PD-1 inhibitor that impairs an interaction between a glycosylated PD-1 polypeptide and a programmed cell death ligand; and
a pharmaceutically acceptable vehicle or excipient.

15. The pharmaceutical combination of claim 14, wherein the pharmaceutical combination is formulated for systemic administration.

16. The pharmaceutical combination of claim 14, wherein the pharmaceutical combination is formulated for local administration.

17. The pharmaceutical combination of claim 14, wherein the pharmaceutical combination is formulated for parenteral administration.

18. The pharmaceutical combination of claim 14, wherein the pharmaceutical combination is formulated as a pharmaceutical composition.

19. The pharmaceutical combination of claim 14, wherein the pharmaceutical combination is formulated as separate dosages.

20. A method of disrupting an interaction between two non-glycosylated PD-1 polypeptides, comprising:
contacting an antibody of claim 1 to a plurality of cells comprising two cells each expressing a non-glycosylated PD-1 polypeptide, wherein the antibody impairs the interaction between the two non-glycosylated PD-1 polypeptides.

21. The method of claim 20, wherein the two cells are located within a tumor microenvironment (TME).

22. A method of activating an immune response in a subject in need thereof, comprising:
administering to the subject an antibody of claim 1 and a glycosylated PD-1 inhibitor to activate the immune response, wherein the antibody impairs an interaction between two non-glycosylated PD-1 polypeptides, and wherein the glycosylated PD-1 inhibitor impairs an interaction between a glycosylated PD-1 polypeptide and a programmed cell death ligand.

23. The method of claim 22, wherein each of the two non-glycosylated PD-1 polypeptides is expressed on a cell and the two cells are different.

24. The method of claim 23, wherein the two cells are located within a tumor microenvironment (TME).

25. A method of reducing tumor cells within a tumor microenvironment (TME) in a subject, comprising:
contacting a plurality of cells located within the TME with an antibody of claim 1 and a glycosylated PD-1 inhibitor.

26. The method of claim 25, wherein the tumor cells are reduced by at least 5%, 10%, 15%, 20%, 25%, 30%, 35%, 40%, 45%, 50%, 60%, 70%, 80%, or 90%.

27. The method of claim 25, wherein the subject is diagnosed with a cancer.

28. The method of claim 22, wherein a combination of the antibody and the glycosylated PD-1 inhibitor enhances the production of a cytokine in the subject.

29. The method of claim 28, wherein the enhanced production of the cytokine is compared to a production level of the cytokine by either the antibody alone or the glycosylated PD-1 inhibitor alone.

30. The method of claim 28, wherein the cytokine is interleukin 2 (IL-2) or interferon gamma (IFNγ).
